SEQUENCE LISTING (16 sequences)

SEQ ID NO: 1 - DNA, 20 bp, Artificial Sequence
  16SrRNA primer for R.leguminosarum
ggagaatgggagtgacctga

SEQ ID NO: 2 - DNA, 18 bp, Artificial Sequence
  Primer for 16SrRNA of R. leguminosarum
cacgtaggcggatcgatc

SEQ ID NO: 3 - DNA, 20 bp, Artificial Sequence
  Primer for 16SrRNA of R. leguminosarum
ttagctcacactcgcgtgct

SEQ ID NO: 4 - DNA, 20 bp, Artificial Sequence
  Primer for NodD of R. leguminosarum bv trifolii
cctgcagttcgcgaagctct

SEQ ID NO: 5 - DNA, 20 bp, Artificial Sequence
  Primer for NodD1 of Rhizobium
cgcttaaaaccgtgctccag

SEQ ID NO: 6 - DNA, 23 bp, Artificial Sequence
  Primer for NodD1 of Rhizobium
gccagaaatgttcatgtcgcaca

SEQ ID NO: 7 - DNA, 21 bp, Artificial Sequence
  Primer for 16SrRNA of Mesorhizobium
aatgggttgcggaagttcggt

SEQ ID NO: 8 - DNA, 20 bp, Artificial Sequence
  Primer for 16S rRNA of Mesorhizobium
cccatctctacggaacaact

SEQ ID NO: 9 - DNA, 20 bp, Artificial Sequence
  Primer for 16S rRNA of Mesorhizobium
actcacctcttccggactcg

SEQ ID NO: 10 - DNA, 19 bp, Artificial Sequence
  Primer for 16S rRNA of Mesorhizobium
cacatggcaagcctcctca

SEQ ID NO: 11 - DNA, 19 bp, Artificial Sequence
  Primer for 16S rRNA of Pseudomonas
gacgggtgagtaatgccta

SEQ ID NO: 12 - DNA, 20 bp, Artificial Sequence
  Primer for 16S rRNA of Pseudomonas
cactggtgttccttcctata

SEQ ID NO: 13 - DNA, 15 bp, Artificial Sequence
  Primer for rRNA intergenic spacer of Erwinia
gaagtcgtaacaagg

SEQ ID NO: 14 - DNA, 15 bp, Artificial Sequence
  Primer for rRNA intergenic spacer of Erwinia
caaggcatccaccgt

SEQ ID NO: 15 - DNA, 49756 bp, Artificial Sequence
  transformation vector
taatctacctgtgacactaagatgatcgaagccactttcgctcaggagttatcacgttaaggcgcaactcataaaaatgcgtcaccgattacttgatgcgcacgcttactatatctcaaatcaaatgtcgaacctcggttgaagcccattgtctacgtcgcgtatatctcccgtattttgcttttgctgccgaccgataccgctgcggttcaatagttgcggaatgtggtgctttcggattcactcaaaaaggagtcgcttatgaaacgtatcagtacaatccttgtcggcgtatttttggctgcgccggtttatgcagccgacaatattcatacattagggacgttgtctgaaattgaactagcgctcactgccggaaagccggtgaacgtcacggttgatttaagtttgtgtgctcccggggtggccgatactccggcgacgaaaacgcaaggaggcatgcgcatagatgcgtatcgaataacaactgacggcacccttgcgtttgcagatcagcacttcaccattgaccgtgacggtaaacccataactcaatttattcgttatcagattcggtcaaacggtgacgccgatttcacgatggttacattcaacatgccaacatatgagcgaaaaggtaccagcttggcctacaagtgcgcgatcgaccacgggttgagttttcgtactccacagtgatttttgcgtccaatctgatccgatgcaagcccaaggtgacgcttgcaattacccacatttgagcgtcgctgccatttcggttctgaggcgaatatctgtcagccggctgagtccacgccagacgaccgtatttcccggaggaggatctgaagttctagcaaggttgccacccaaacgagcgagtttggtgagatagaagtttagcgattcgcaacgaagcacgaccgcagaaccacttacttcgaaggcttcgtatatatcgttcctgcagtgatctggctgcgatgaatgtcgattcgtcctggtggtagcatcagggatagtcctacgacgaagcataggatttaagtcatcacgtctgttaaatgccgccggcgctggccacgcgaagacaaagagcgcctcgtcgctgcctgctttgagccgggcgcagttatctctgagattgcctgcgcggacggtatcaatgtcagtcagtcggaagcatagcgatgtaataatcaagttgggtcgggaccgacgtgtccgcgtggatagcaacatcgacacggaagcacttggccgtattctcgattgcgtgctggggctgcgatgatcatggtccctgctggtgtgaaggtctggcaggcaacgggctccaccgacatgcgcaaaggcttccccggtctgtcgttgatcttgcagaaagcactgatgcctgacccgaggtgtgggcactgttcgtattatacggtcgtggcggccaacgaccgacatactgcatacggatatcgtttcgcgacgatctcctacagccgtcaaagctttcgagcgcaagcgcccatcacgcaaactcttcccggttcatctgtcgcgtgagcgtgtcgttatcgccacccctacgaactgtgcttgctgcgaatccgccaaactgtcatagcttggcgaggatgttaccgagacccttggaagtcatccgagatttggcggcggcaacctctgcaaaatgcgttatgagcctggtttttcagccacagacaagtatcgcgttcgtgaaaaagcgcacgcgcgatagttctttagccgcacctctgacgccccatttacggccggatctttgcctgaccttgcaaagtcgactccatcgccttacggcacgaataacaacgtcgcgtttcatcgacgtcccatcagccgcttgtgtgttcaattcttctctgtttcacttgaaacaaattgaatagatattcccgctttcaaagccatttacaaatcctctcgtgcagcctaacgccattggcgtggaacaagcgttggcacgaggaagtaagtgcgatgaacggaaggtattcaccgacgcggcaggattttaagacaggcgcgaagccttggtctatattagcccttatcgttgccgcaatgattttcgcgttcatggcggttgcgtcctggcaggacaatgggactacccaggcaatccttagccaaatacgatcgattaacgctgacagcgcctcactgcagcgcgatgtactccgcgctcacacgggcaccgtggcgaactaccgccccattatctccaggctgggagctctgcggaagaatctggaagatttgaagcaattatttaggcaatctcatattgtaagtgagagcaatgctgctcaactgctacgccggctagaagtgtctctaaattcggctgacgcggcggtcgccgcctttggtgcgcaaaatgtccgcctgcaagattcgctggccagtttcactcgtgctttaagcaatcttccaggaaaggcctcaagcgatcagactttagaaaaaccaacagaattggctagcatgatgctccaatttcttcggcaaccaagcccggctatttcattcgagatcagccttgaactagagaggctccaaaaacaacgcggtcttgatgaagctcccgtgcgcatactttcgcgtgaaggtcccattatcttatcgcttttgccacaggtgaacgatctggtgaacatgattcagacgtctgacaccgcagaaattgcggaaatgttgcagcgtgagtgtttggaggtctatagcttgaaaaatgtggaggagcggagcgcacgtatctttcttgggtccgcttcagtgggtctttgcctgtacatcatcaccttagtctataggctacgcaaaaaaaccgattggttagcgcggcgtttagattacgaagagctaatcaaagagatcggagtatgttttgaaggtgaggcggccactacgtcgtccgcgcaagctgcacttggtattattcagcgcttctttgatgccgatacgtgcgcgttagctctagtggaccatgaccgtaggtgggctgtcgaaacattcggtgcgaagcacccaaaacccgtgtgggacgacagggtgctacgcgaaatagtctctcgtaccaaagcgaacgaacgggcgacggtattccgcatcgtatcgacgcaaaaaatcgtacatttgcctcccgaaattccaggtctttcgatactactggctcacaaatccacagataaactaattgcggtttgttcactgggttaccaaagctatcgccctcgaccttgccaaggcgaaattcagcttcttgaactcgccaccgcctgcctctgtcactatatcgatgttcggcgtaagcagaccgaatgcgacgttttggccagacgattggagcatgcgcaacgccttgaggcagttggtacacttgccggcggaatagcacatgaatttaataacattttggggtcaatcctcgggcacgcagaattggcccaaaactcggtgtctcgaacatctgtcacccgaagatacattgactatatcatttcgtcaggcgacagagccatgctcattatcgatcagatcttgacgctgagccgaaaacaagagcgcgtgatcaagccatttagtgtctcagagcttgtgaccgaaatcgctcccttgctacgtatggctcttcccccaaccatcgagcttagtttcagatttgatcaaatgcagagcgtgatcgaaggaagcccgcttgaacttcaacaggtactaattaacctctgcaagaatgcttcccaagccatgactgcaaatggtcaaatcgacatcttcgtcggccaagcttatttaccagctaagaaaattctggcgcatggtgttatgccacctggcgactatgttctactatctgtcagcgacaatggtggaggcatttcagaggctgtgctaccctacatttttgaacccttctttacgacacgtgctcgcaacggtggaacgggtctcggcctcgcttctgtgcatggtcatatcagcgcgtttgcaggttacatcgacgttagttcaactgttgggcatgggacgcgctttgacatttatctccctccgtcttctaaggagcccgtcaatcccgacagctttttcggccgcaataaggcaccgcgtggaaacggggagattgtggcacttgttgagcccgatgacctcctgcgggaggcgtatgaagacaagatcgccgctctgggatatgagccggtcggttttcgtacctttagtgaaattcgcgattggatttcaaaaggcaatgaagccgatctggtcatggtcgaccaagcgtctcttcctgaagatcaaagtcctaattccgtggatttagtgctcaagaccgcctccatcatcattggcggaaatgatctcaaaatgcccctttcaagggagaatgcgaccagggacctttatctgccgaagccgatatcgtccagaactatggcgcatgcaatcctaaccaaaatcaagacgtagagttgcgacgtatcaggactgggcaatcagatttcggttcggtggagcgcgacaggggaactacatgaaagatcgaacggcataggttcaatggatcaagccgggcgaggcggcttgatttgtccgatcaggtccggtcgtttggttgaaaggatttgcgcaccaactatgacaccaccatcattggtgccagcagattaaagtaggcctgaaccggtgcggtcaaaaatcggggtggacgtcggctattgtacaggtcagattgctgccaaatgccgacgcgggaattgggcacggtgtcgtaagcgtggacagagattacctcgtcgttgatcgaacacgagagccatctgacgaacaccttatgccgccacccattaacatccactgcgattctcgtcttcttcagcacgagcatgaagtctcaagaagtaaacttcgagcgttggtcagcctataagatgagttcgccagaaagggcaggcgttcgcgcctccctcaatgcagaaggctacttccattgtggtcggcaactgccaggatgggaactctcccgataatccaaccaacgacagcgcagtggtaaagtaagcggcaagctgaccccatctggcgtcagtagcgcgggctcagctattgcgtaatgactatccaccagctgtgagcttctatgtctgcgcctatctctggaactagaaactttcatatgatcgaggccgttccggaacgtttcgagggcgcgccgcggcagtttcggcggcgttggtccgatgacttcaaggagcgagcggtggctgaggcaatggagcctggagcgagtgtctcggcaattgcgcgtcgcatcggcatacatccatcacaattattcggctggcgtcgtgatgctcgcgccaggcaagggggctcttcggtgcctgcggctgtcgaggcgtgtttggggccagtttgtgcgcgggcggtgatcgaggtggttatcggcgacatgattatccgggcggggatggatgccgatgaagcacacctcagtcgcgtgatccgagtggtgcggtcggcatgatcccgtcgaacgtaaaagtctttctggcgagccatccgatcgacttccgtaagggacctgacagcttgctatcactggttcgagatgctggcagtgatccgttcaacggagctctttatgtcttccgggcgaaacgagcagaccgtatcaagatcgtatggtgggatggctccggcgtttgcctttacgccaagcggctggagaaagcccagttctgctggccgcgcatcggacatagccgggtccagctcaacaatgcccagcttctggctttggtggatgggatggactggaaacgcgttcgatcaacacccatcaagccgccagagattgttgggtaaaacccctgcggcgaattgaatcatgcacctgaaactgcgggcggatcacagcaaaatatgctctgatcatacccatgacgcgacccgatatagagctcccggatgatgtagaggcactcaaggccatggttcttgccatggccgaaaaggccgctcgtgtcgaggctttggaaaagcaggtcgatgatctcgaggcccgaagtgctgacgccgatgagcgcatcgagcggctgacacagatcctgaaagcttttgaccgcgcgcgcttcggacggcgttcagagaaacttgcagcggccagtgccgatgacgagcagcatgcttttgtctttgaggagatcgagaccggtattgcggcgatcagggccaaggttacgagtggcatcaacaaagatggcaaacgtccaccgaggccacgcaagggctttgcggcgcatctggagcgtatcgaggtggtgatcgagccggaagacttgccggagcatgcgggtaagaccaaaatcctgatcggagaagacatatccgaacgcttggatgtcgttgcagcgaagttccgtgtcattgtcacgcgccgtcccaaatacgcttttaagaacgaagatggcgtgatccaggctgcggccccgacacatatcattgaggcgggtatcccaaccgaagcgcttctggcccagatcgccgtctccaaatatgccgatggcctcccactctatcggcaagaagcgatctacgcccgcgacaaggtcgaccttgaccgcaggctcatggctcaatggatgggaaagctggggttcgagctcgacatcctcgccgattatatcctgaacgaaatcaagaaggcggaacgaatatttgccgacgagacgaccctgccgacactggcgcctggatccgggtcggcgaaaaccgcatggctatgggcttatgccagggatgaccgaccgttcggaggcaacagccctccaatggtggcctatcgcttcgaagacagtcgggcgaacgactgcgtggcacggcacctcagcggctatcgcgggattctgcaggttgacgggtacggggcctacagcaagcttgtccgcaaggacggcggcaacgacggcgttattctggctggctgctgggcccatagccgtcgcaagttctatgaattgcatgtcgcgaaaagctccaaggtggcaacagagacagtcgagcgaatggcaacgctctgggaaatagaagataccgtccgcggccaaagccctgaggcgcgtgtcgctgcccgacaagaccgctcggcggcgatagtccgggacctcttcactctctggcagacgactttgccgcgagtatcaggcaaatccaaactcgccgaagctctacggtactcgatcaagcgtcgtgacgtcttcgaacgctttctgaccgacggccgtgtcgagctcgactccaacatcgtcgagcgagcgatcagaccgcaggcgataacaagaaaaaatagtctcttcgccggcagcgacggcggcggcaagacatgggcgacaatcgcgacacttttgcagactgcaaaaatgaattctgtcgatccccaggcctggctgacgcagacgctcgagcgccttgcaaacggctggcctagcagcgaaatccatacccttatgccgtggagctacaaggcctgaacggcctcagcttgccgcttacgtggtaaacagagccaccccatcaggaatatcgcccattccgtcatcagcgtggtgacgtgcatcaggcgcgcttgttgcagaactttaagtgcttctttcgattcgagatctcagcaaaagttctccgcaaaccttcaccgcgggcttacgtgtcttttgtaacaaattgcgacgcagttgatatccacttgaaacattagtccgaaattatcgagatctccgctgataaggtatcgaaatggcgataaaattggtattgatactcgtattcacactctttcccgcggcagacgctgcatatgcgaatgaccgcgccaacggtttcatgtggtcaaacgggggcgaaactggagtgaggcttcctcttcgggtgttcaatgccaagccagccaagaacacggtggcgatcatttattccggagacgctggatggcaaaatatcgatgaggcgattggtacctatctgcagacggaagggattcctgtcattggcgtcagttcacttcggtatttctggtcggagcggtccccaagcgaaactgctaaggatcttggtcacataatcgatgtctacaccaagcatttcggtgtgcagaatgttttacttgtaggatattctttcggcgcggacgtcatgccggcaagcttcaataggcttacgcttgagcaaaaaaatcgggttaagcaaatctctctcttggcattgtcacatcaagtcgactatgtcgtctcatttaggggctggctccaactcgaaacggaaggtaagggcggcaatcctctggatgatctcagatccattgaccctgcaatcgtccaatgcatgtacgggcgcgaagaccgtaataatgcttgcccatctctccgacagaccggcgcagaggtgataggcttcagcggaggccatcactttgataatgatttcaaaaaactgtctacgcgcgtcgtctcaggcctcgtggcacgcctaagtcatcagtaatctttagttcctgcaccgctttagtattgactgggatagcgacgcctgtgatgcagacatcggatattgtgtcgttaagtaaaaggccttcgtctgatcgcgagactcgctagtggttttcaggtgagtgagatgttttgccgcaagttgcgctgagatcgcatctgcctgcggctgccgcacctccagattggcagcaacaagatcatccttcaagggaagatgcctataacgcatgtgttgacgactttcgcctcgtgaatgatccggtctgttcccgacagtgggatgccgtcgcgatagagcaggctctcgcgccaggaggaatttctccgatatgacttctctccttctacgtgcccaaagcagcaacgaaatcatctatcgcggccaaaagagcggtcatgtgaaagcacccttgccagtcaattccctggccaaggtcagcacggcgatactgcgaggtgttgctacaccacggaggcgatctagaaggacgatttcatcatttaggccgcacactcataaatcaggtttgcaaatcggtctgattttgattcattgaggcttgacttggaggccactgataacccgtccccgctttgatgtcaccgatttcgaatggactgttgttcagtcctttgttcccaaataagccgcgtcgcgtgccgcgcgttgacgcgggtgatcaatgtcatcttgtggcgcttccggacgggcccgccctgggcagacgttcctgatcgatacggttcctgtacaacctgctacaatcgctttgtacgatggcgtgaggcagacgtctgggatcatattctgagtgtgatttccaaggctttcgatagaatgtcgtcatgattgacagttcctgtgtccgcgtccaccaacatgcggcgacgggaaacagcgggatcaagacgatggctgtatgggacgttcccgtggcggtttgaccaccaagattcacgctgttgtcgatgccgacggttgaccgatccgtcttgcgctgacagcgggtcaagcccatgaccgccgcatgaccgaaacatttttacaaataatcgcttagagtgcgtttctgctggcggacaaggcatatgataccaacgcgataagagcatttgcaaagcgcaagcaggcatgggccaatattcctgccaagagcaatcggaaggaagcttcccgttcatcgaatgggtttacagacagcgtaacctcgccgagcattttcgttctgagcctgacctgaccaactgcggcctgaataggtcgatccggttgcttagtcatggatgcgcggttctcggtccatgttgcgttccaagacgccgggcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcgccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttggctcttaattatctggctcaaaaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtcatgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggctgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatagcctgcgaaatgatcttaatggtgcaattcgtttcaggcggcatgttgcggttcaacaaagtgtacgccagatcccaattggccctttacgaggtgtcggtatgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctgataagtcgccaatatagtgatcatttgcagtattcgcatcgatcgctggttgatattctgccgctggtcgaccggctgctcgtcgccaaaaatgctcacagggatacgatggcctcggtcaggccgcgtgcgtcctgtctttccagttcctccctttcagctcgattgtggcatcatttattgcctgctcattgcagttgaaacgcgatatccgtttcaagacccgggtatggatggtactttggagatatgagcacttgccagacttcccggtcggagacttaaaaattccgataagaggttacggacgacggaccttataagtggatcgtctagtggtggcgccgatcaaaacagttccgcccccggctttgctcaaagtagcaaagcagctttatctcgggttgcggaggattttctagaaaaccgcaattttgcaggagagaacatatggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtgagacgatggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttgacgttcctgctgccgcgtcaacgaggaagctcgtttgacccgggtttgccaatgaaagggctcaatcatggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaaggtggaacgcgtctaggcttgcctatcatcaccacctcatgatggtcgcggagaccttcgaagagatatccgacatcattggagaagctgggccggaaactgggcttcttcttgacaccggacacgccgcggcagccggattcgactacgcgaagctcatcgagcggttcggcgaccgcatcgttcatattcatctgaaggacgttcgcaaggcaagccgcgccgaggtccagtcaaaggatctcccatcggttgacgaaaagacatgattttcgttaagcgcgtcgcagggcaaggccgcaatcgcccgcagcaaagggccggcaattgaggttggccccgctaccccctatcagattgtcgccgacatcatcagacgagattggcaaaagctgcagctggtatgctgctatgcggcaaatcaagacggtggtcgatcgtctcgagcggttgagtgcggcggccagagcgagccagaccaactttttgtcgaactgttcgcagaagatgggccgcccgcagggaactgcatcgcaccagaaccgacgcgaccatcaagtctgctcgcggaccgctgaggtgcaggtcagcgggccgacgcagatatcggctttgaacgttggctcccgcagacggggctcgcggggtcgtcgcaaagacttctctgtacgtattcgtgatgagatgatttgtctctgtatgcagattgcgatgcgggatcaaactgcaatcccccggaagcacaatgggcctgcatcaggatgatcgcggctcgatcggaaacacaagcagtgtcgaggttgcggacgcacagagccggccatccgagtcgactagccgcgcttcggagaaggcaacccgtcgaccaagtgacacaatctttccttcagcccggaccttcccgctgctctcactcaaggcgcgatgatacgccactttcaattcaagcgtcgtatagccctggcctgccggaaggaccgaatgcacggcgcagccacaggcgctgtcgagtagtgtcgcggcatagccgccatgcaccgagccgatcggattgaagaccttgcgtgacggcgatccttcgaacaccacccgtccgcgctcgagttcgacaagagcaaactccagcgtctcaccgatcggcggctgaataccggcctgcatgagggccgccaattggtcgtgaccgtcgaggccatggttgctttcaaggatgttcatgccgcacgccttttctgcgcggggtcggcgcgttgggcgtctttttttcgtcgatccacacccgcgtctcggacaggacctcgtccggcagtgtggaatcatcaatcgaccgggcgaggatgagcgcaccaaccatcgcagaccagctgccgatggcagctctacggcgtttgacaggatcaacacccggtatcatctcggtcagagtgtcaatctgcgcggctagaacctgcgccatcgaagcgcgtgcctctggagcctgttgccgcatgaaacccgcgagagcggccatggggcaacccatatccgcatgctcacgatggggtttcgagagataggtgtcgatccaggcgctgagatcgccgatccctgattgactgcctgcggcatgcgcgatggtttgggcgaccagatcgtccttcgatcggaaatgcccgtaaaagccgccgtgggtcatgccggcggcgttcatcacttcggcgacgcttaccgcatcgaaacccttctcgcggaacaggcgtcccgcctcggtcaggatgcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaggatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctggtcaccaattcacacgtgatggtgatggtgatggctagcgttcttgtagccgaaatctggaatgttggtccagcgctcgcgaaagacgtgcgcggcgagcttcggcttgcggtcacgagtgaacacgcccttcttgtttccttggacgcgcatcacgccctgagaggtcgcgaagtccgcgaagttccacgcttgctcacccacgaagttctcaaactcatcgaacacgacgtggttcgcctggtagtactcgacttgatattcctcggtgaacatcactggatcaatgtcgtgaaagcccgcaacggtgtctgcgccgtactcagtgatcatgatcggctttcctgggcaacgcttgttccacgcgtgaaattcctggcggagatggactttggccgcttcgagatcaccgccatcgaagtaccatccgttatagcgattgagcgcgatgacgtcaatcagttcggcgactttgtccgtctccggggtagccatcacaaacagcacgatcgtgaccggacgcttctgtgggtcgagttccttggtcagctccaccaacggcttgaagtactcgtacgcgccctcttcctcagtcgccgcctcgttggcgatgctccacatcacgacgcttggatggttcttgtcacgagacaccagttcacggagaacgtcttgatggtgctcaaacgtccgaatcttctcccaggtactgacgcgctcgctgccttcgccgagtcccgtggtggccatgaagttgaggtgcacgccaactgccggagtctcgtcgatcacgaccagaccctcgcgatccgcaagacgcatcaactcttcagagtacggatagtgtgcggtccggaagctgttggcgccgatccatttgaggatattgaaatccatcacattgctcgcttcgttaaagccacggccgttgataggagtgtcctcatgtttgccaaagcccttgaagtagaacggtttgttgttgatgaggaacttgccgtcgttgacttccacggtccgcacgccgaacggctcttcatagacatcgatggtcagtccgtcgttcaccagttccactttgatctggtagagatacgtgttcagtggttcccagaggatgacattcggaatctccacgttaccgctcaggccctcggtgcttgcgaccactttgccttcctcatccacgaccgacactttcacggtctcggctttgccttgaaagtccaccgtataggtcacagtcccggttgggccattgaagtcggtcacaaccgagatgtcctcgacgtacgtaaacggggtcgtgtagattttcaccggacggtgcaggcctgcatagttgaagaagtcgaagttcggcttgttacgaatgacttttccgaggccctcttcgtggcgctcgctgtacagccccaccgggagggtgctatcgtcgaggatgttgtccacggcgacggtgacgcgattcatgccatcacgcagcgagttgttgatttccgcttcgaatggcaggaatccgcccttgtgctccacgaccagctcaccattgacatagacaattgctttgtgagttgcagagccgaagcggagcacgatacgctgatccttcagataggccggcaccgtgaactcacgttcgtaccagacatatccgatatggttgcggatttccttggtcacgccaatgtcattgtaactgcttgggacggccatactaatagtgtcggtcagcttgctttcgtaccacttctcttccagtcctttcccgtagtccagcttgaagttccagacgccattgaggtcgaagacgccacgggtctcggtgttgatcgggtacagactagttcgtcggttctgtaactatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttaccctcagatctaccatggtcaagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatgaagctaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcctgcaggcctaggatttaaatggtctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcacc

SEQ ID NO: 16 - DNA, 42259 bp, Artificial Sequence
  transformation vector
attgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgggatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgaattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgcatcctgaccgaggcgggacgcctgttccgcgagaagggtttcgatgcggtaagcgtcgccgaagtgatgaacgccgccggcatgacccacggcggcttttacgggcatttccgatcgaaggacgatctggtcgcccaaaccatcgcgcatgccgcaggcagtcaatcagggatcggcgatctcagcgcctggatcgacacctatctctcgaaaccccatcgtgagcatgcggatatgggttgccccatggccgctctcgcgggtttcatgcggcaacaggctccagaggcacgcgcttcgatggcgcaggttctagccgcgcagattgacactctgaccgagatgataccgggtgttgatcctgtcaaacgccgtagagctgccatcggcagctggtctgcgatggttggtgcgctcatcctcgcccggtcgattgatgattccacactgccggacgaggtcctgtccgagacgcgggtgtggatcgacgaaaaaaagacgcccaacgcgccgaccccgcgcagaaaaggcgtgcggcatgaacatccttgaaagcaaccatggcctcgacggtcacgaccaattggcggccctcatgcaggccggtattcagccgccgatcggtgagacgctggagtttgctcttgtcgaactcgagcgcggacgggtggtgttcgaaggatcgccgtcacgcaaggtcttcaatccgatcggctcggtgcatggcggctatgccgcgacactactcgacagcgcctgtggctgcgccgtgcattcggtccttccggcaggccagggctatacgacgcttgaattgaaagtggcgtatcatcgcgccttgagtgagagcagcgggaaggtccgggctgaaggaaagattgtgtcacttggtcgacgggttgccttctccgaagcgcggctagtcgactcggatggccggctctgtgcgtccgcaacctcgacactgcttgtgtttccgatcgagccgcgatcatcctgatgcaggcccattgtgcttccgggggattgcagtttgatcccgcatcgcaatctgcatacagagacaaatcatctcatcacgaatacgtacagagaagtctttgcgacgaccccgcgagccccgtctgcgggagccaacgttcaaagccgatatctgcgtcggcccgctgacctgcacctcagcggtccgcgagcagacttgatggtcgcgtcggttctggtgcgatgcagttccctgcgggcggcccatcttctgcgaacagttcgacaaaaagttggtctggctcgctctggccgccgcactcaaccgctcgagacgatcgaccaccgtcttgatttgccgcatagcagcataccagctgcagcttttgccaatctcgtctgatgatgtcggcgacaatctgatagggggtagcggggccaacctcaattgccggccctttgctgcgggcgattgcggccttgccctgcgacgcgcttaacgaaaatcatgtcttttcgtcaaccgatgggagatcctttgactggacctcggcgcggcttgccttgcgaacgtccttcagatgaatatgaacgatgcggtcgccgaaccgctcgatgagcttcgcgtagtcgaatccggctgccgcggcgtgtccggtgtcaagaagaagcccagtttccggcccagcttctccaatgatgtcggatatctcttcgaaggtctccgcgaccatcatgaggtggtgatgataggcaagcctagacgcgttccaccttagaaccctctggaggtggaacgctctcttgaacgtgaaccttcgaaaatcatagacctcgcgcattcgggcttcggtgaaggaaccggcagtggcacactaagcaattgattcagcaaattttcctgttccgaagctgacaaatggtaatcttccgatggaatttctggccccgattttggacctctcgataagtccggttcaccgaacaacgattgcgggtcaataaacccgttagtagtattgatcgaataatcccgtaccaaatgcccttgtacatcttcgtcgaagatactctccgacattgaactcgttcgccctgagctacctctgaaagagccgccgggcgcctcccccgcaagtcgctcttgatcatcatgtattgccacagaaactggggctggcgcattatgaaggaaacttaaaagttcttgctgtcctggagctggctgatgattgtccgaggatgaagacatcaccggtccttcgcaagccgtttctccaaccgccgtgtgcagatttccactcgggtgtgccggctctccccgcgagccaaacaccttagttatttccggcgacccgggatgcggtaacaccaacggctctggctgggagaaatgtcgcagccgctgatcattctccttgtgcccgtcagattcattatcgcgccggagggtgagatgagctaacccttcgtatgggcgaggtccgttcgcttgaaggtctggatttgacaacagcatgtttgctggcttgtatcgtgattgagaatctaaccatccttccaagttccgttggggagacgatgatgtttgtccagcaacatcaacatgttgatctgtcacacgcgtgacgtcagcttcgacgtgatcagggttggctttggctaggtgcgactgctgtgcgaaatgaatgtcatcagtctccgggaggggcgaaccgggaatcgctgcccgctcctgccagatctggcgatgcgcaccggattcaaaactacgcagtctctcagcctcggattctcgctcgctgagcgccgcaaatttccggtagaccgtcaccccgagtatcttttttgaaaccttcttgaggcccgcgctgagatattcggtttcgctcctgtagatcttgccgaaggtatgcttggcactcttcaattgtccggtagcttcatcgtagacagcagaccgcttgttatagagccgtcgatggctcaaaatatcgcggcccatttcattgccctccttgtccagaagggacctaaaagatttgttgaaagcgccaccaagtttttgaatcttcgtctgggaggtttgacgattgggtgccttcactgaaatcttggaaaagccgacatggttgctgccgatacgctcgaggtttccgtgatcgtcccgttcgaatgtctcccgtctgaaaccgtttcgacgggttagatcgcgatagagccggtcgtcagggccgctccgatacggcgcgcccattttctcggatacaggttgaaagagcctgccacggttggcgtaccttgtgcggatcaggtcgccattttcgtcgcgctcccatttttcctcaaaacgtccgtctttgtatttcacgtgcttcatgcggagtgtgccgtccgcagcatacaaatggctgctgacacttaaccaggacttcctttcatataaacccaaatagacttcctgcgtcaccgggttacgcttcagaaccgttttggttttgcgaagtctaccgtgctcacgcgttaattctttatggttgtctactcgaagccggttatttccaagaattttatagcctatttcgcctcgaaaccctacgtcgagtttgtcgactatcgccatatcagcagcatactttcgcttcctatcgggactctccagtctggcttgccgcttgagcgcctcttcttcggtaatcaatgcttttcgcatttgctcgacaacgcgctgagcagaacggcgcatatctgccgtaagcgacttcgcaaaacttttctttgtagtgttcaccatgattgagccctttcattggcaaacccgggtcaaacgagcttcctcgttgacgcggcagcaggaacgtcaaaagctgttgacgctttggctacgcggtcgactttcgtatatcctgcggctgtcactgattggacgcgacgccggctctggccgaacgaaatcacgtgaatatttgtcagttgcttcaccaagcacccccgctgcatcgggtggcagccttaactgcaagcgctcatattccgcgacgctggtgtacgtgccagttctctgaccattctcattgcgatcggcgacgatgacatctttggtggacaaattttgcagtttttcaggagacaagtcatgaatgtcacgtaactctgggccatttaagacaataaccgctgcctgcgcgtcaaacttttcgaatttgatactagccgcatggctttgccgacccatcgatgcgggtaaaccaacccggttcatcaactcggtgaaatgtgcagggtcagaatattccaatctgggcggcgacagatagcggttgccgtcgcgagcaagcaactgaacgcgatcccacagttctccctttttcaaagcttgcgcccaaggcgaattgggtgcgctttgcattaggaagtcaacgctcacatgacggtcgcgggtcaaaccgccctgcccgtctggctgctgtcccatatttactgtcgcagttggaaactgccgattgtgagctctattttccccaacgaattccgcgaagcgaatatccgcactgccccgttcccaggattccagatatttcgagtcatgcataatgcctgactttgacttcaatttgatctcagtctcgtcgccatacctgcgcttgacggtgtgttcaaactcgctgaactgtttgttggcgtaattgtcgccggcaaacctgtacacgaagtggatgtcattttggggagttttgatcataatatcgggcagcagcgaaccagcctggagttcgtgttgatatcgcttttgaatttcccggcgcccgtatttttctgtttgtacgtatcggtcttcaggtcttagtttataattccggtcgagctttatgttcttatctgtttgataaaagatctctgtgcccgtgatccccatatcagcgtgcgcatgattccatacgtccaacttgtactgaaaccgcagtttgttacgttccttcgcctccatcatcccccatttttcctccagttgctctggaacgctaatcttgtcgaatgcgagaggtggcgggtagtaattaccggctccgtccctgtagagacggcagcacgcgttaaattccgccttggtgggcatttggtcgaggttgcctttggcaataccagtaaaagcatcttgtcccgtttgatgcacgagaatgtattcgtccgggcgccgatcgttgcctggcaaagacatgttcgagaacaagacggacagctctttttgaatctcagcttgatttccgtgctcgctgccagaataaaggctggaggaggagtcgaccgatccataatccagacgtgggcttacaacttccgttcgggtagggctccctgattgtacgtcttcgccattcgtcatctgaatatcggagatggtgttggaactgatattcgccttcttccaacgcctggggtcagtgtcatcatcattttcgatgttgtgaccgcgacccacatagacattctcattgctagacggatccatcgtctcactccttctgaccagcaagattgtcttctgggagcgggcaccgatggacaaagtcctccagctcgatcatagcgagatccaggttggtaaaaccactcggcggatgactgccgctcatttcctctatgagagactccggtctctctatcggcgaggttgtcctatttttgttcacatgcggcttgatgatggccatatgttctctcctgcaaaattgcggttttctagaaaatcctccgcaacccgagataaagctgctttgctactttgagcaaagccgggggcggaactgttttgatcggcgccaccactagacgatccacttataaggtccgtcgtccgtaacctcttatcggaatttttaagtctccgaccgggaagtctggcaagtgctcatatctccaaagtaccatccatacccgggtcttgaaacggatatcgcgtttcaactgcaatgagcaggcaataaatgatgccacaatcgagctgaaagggaggaactggaaagacaggacgcacgcggcctgaccgaggccatcgtatccctgtgagcatttttggcgacgagcagccggtcgaccagcggcagaatatcaaccagcgatcgatgcgaatactgcaaatgatcactatattggcgacttatcagcgtttaaacgctttgtcttgagctcgatcacgcggatcataaactcgtcgatcatcgagctcatcaatggctttttcagtctctgctttggatcgagctttcgaaggcgacgtgcttatcaagcgcgcagttcggtctaacgcagaaataacttctttttttttccgtcttacgccgactgatttggcctcaagatctttttttacgtcatataaggccaaaaactcgcgatacacttgctgctcacgctcgagagtcggccattttccagtcaatgctgtttcgcgggcaagaacagcttcggtttgaccacggggaccaagatgcagcaattgaggggcttcgtttttatccggcggaacaaggacaggatcgcggctcactgtatagttgtagctcgttgttgccacttgaattggcaaggcaacaccatcgggcgattcactttgcaggccatcgctagcgagttggtgcgcgtaacccaattggttcaattgatacccatttgaggccgtcgcgccttccgagcgcgcaacggggtccgacagagttgccctttgatccgagcgatagcttgtgtcatccggccatccgcgcccgacaagtttggtccgtcctctcccggtgccaatatgttggcgatgaagccccttatcctcaaggtaggcgggcatgggcctttcgagacgccggccctctttcgtcgctctcgcagtatcgtggacagccttgaacccgaaaatatgctccgactcatacgcaccggtttcggtcgcgagccgcgctcgttcatttttccggctcccataggtgccaatattgtcgcttcttaagcgaacagtcttttctggcgaagggcgcggagcctccgatccaaattcgttaagcaacagtcgccttgtgctggaattcacgtcatggatagaagctgtaacggttctgtcgcttttggagcctagccgcttaactccacccgtgcgatcgacgggatcgctggcggtgatattttccgtattgtttgcatctgacccagaagcagtaaaccattgcacaggcctcgtttcagattctgaacatgctgcgaattgcctttgagacgattctggcacctgctcccttactaggtcggaacgacgatagattgattgatcgtctaaagggacgtctgatctatccgtcaagcttgtgtacgacgggcgaccaagcgcgtcgctatcatcaagcggatcgcgctcatcggcaatccaggctgagggatcgatttctgccagacgagcgttcactacaagattttgccatcgcggttgggctggctgccgccatttttcgtaagcctttcgttccctgtctccgagcgactttccttcatataagaaaaccggtgaggacgactgaccaatcgcttgggtcgtaacagcgtcactttcagtcctggcctggtgtgcatctaagatgagatcttcccaagtcgggcgttccctcttactccagcgttcataagcagttcgctcaggtgctccgagccttgtgccatcatacacaaaagaatcggaggaatctgtccgttgaccaatggggcgttgcgaaccgcgttccgacgcgggcagagcctccccgcgctcttcaaagcaacctttttgaccttcaatccaaccagcatggcaaagttctgccatacgcgcgttaacaacgagatcttcccaggtaggctgagcgtctccttgccattgtctgagagcctgccgttccggctcgcccaagggaacaccgttgtattcaaatgatgatgaagatgtttgtgaatcaaaaagccaagcagagtcattgatcgaggaaacgcgtgccctcaataccaaatctttccacgtgggtggctccgcctcggcccagtcaccatatgcagaacgctcggcttccccgagcttcatcccgtcatatgaaaacgatccggatgaattctcatcatcaattaagttgctccgctttcgcttcaatgaaattgaacttgtattcttcgatcctgcgagggatgtatactcgccggtaatcccgtgtggacggccaagttccgtcaagcgtgcgctgacaacaaggtcttcccatgtcggctgagcgtcctcttgccaattttcgtaggcctgtcgttccccgatgcccagaggcatcccttcgtatctgaaggttgagggggtgtcctcttctaaatctcggagccaggtcacatcgtcaatcggatcgacacgtgccttgattataagatctttccaagtgggccggcccggttcgcaccagttctcgtaagccgcacgctcggcaggaccaagtccaacaccatcataggtaaacgacacggaagtagactggctgtagtcggtttggacctcaaacgaggcatcaacctgatcccgagaaggttcggttaaaaaaggggcggtagtacttccgccaggaacgtcaggaagcgcgtcagccgaacctcttatgtgaactttcgactttcctgtcataccgacacctcgtaaagggccaattgggatctggcgtacactttgttgaaccgcaacatgccgcctgaaacgaattgcaccattaagatcatttcgcaggctatgccggtgcggacctcgatctttgctgaagtgcaataatctggtccaacaattgctgttgagcggtcagagcgagcgtcaattctggaggcacaaccgattccggcggtatctcgcccgcatcggcttgttcctcgtcaccatttattcccatcacgcgttcagggatatcagcaccgactttaacacttccatggtcctcggccacagttctgttttgcggtgcagcatcctcattaaaatttgctatcggagccaagtggtattgaacttgatcgttgctatagtcggaaagcatcaagggtgcgggctcaggcagagagcccatctggcgttcgaaaaggcctttcagcgtacgatcggaataatactgcacctttcgtaatttgagtggaggtcgccctttgatgagaacgatttcactctgatcgtctagcaggcgcacttgttcggggcgcaaaagggctgcaccttgatcagaaatctggatgttgtggtcgaacattctggcttgactgtaagaggtcgaacgcgctttaaacgtatattcgccgatagcttttgagatgtaggttggagtttcgtcatcagccgtggccataaatacttgtacgccagtattgctcagaaagttttgttttcctgcgtcatcgtatgtgcccgacaaggccgaaagactttgaataataaacataaagcggcctctgtaaccggcgatggttgtgatcgcggtctctatggcctcaagcttgcccaggtgtttgaattcgtcgaggagaaataaaacttcatggcactcgtcttcacctggcagcgatctctgcaagattgacacgagctgctgaaacatcaggcgcataagtggtgccaagacctccagatcgttgggactgacacaaagataaatgcaggtcttcttcctccggagatcgtaaacggaaaagtctgaccggcttgtcgctgctttgataagcggatcaacccacaggttcagtccaccgtcgcccagcacagaggtgtacgatgtcagaattttcgtgtcgttgcccgccatattatcgaagatacgctgagcctctttgtttaggctttcctccgcgagttgcgcaaaaagcttatacttttcgccaggctgcgcaaataggtcatataccgcgccaatcgttggtgtgccacgctcaatgcaggtaaggattcccgcgacgaacaggtcacgtgcgccgtcaataaagccttctgctccctttcccttagccgtaataaggttcgcagctagacggcgtgtttcagtgaactggcgttcgggcggaagtgcggcaatatccaggaccggattgtaacaatgagtcttccgctcaggatctaggggggagaacttgaaaactgcgtcgccgctcgctttgcgtgctctggacgtcagttcaaacaattctcccttgacgtcaagggcgattaccgagcccttgaaggtcaacagcgttggaatgacgatgccgacgcctttgccagcacgcgttggcgcgacgacaagactgtgtggctgttcgccattggtcaaataactgccgaaccaaaggggaccacatgtctttccaaagatcggccccttgatgcgactgtaacgctgcagataaccagcatgccgcatttcgccaatttctgcccaacgagctgtcccatgatgcttctgattgcgcagcgatatgatgagctgacttgaaagaacgatcagcgaggtaaagatgacaacagataaaccacgccagaagacggtgctggcataacccaagtaaagcggggtctcataccaaaaagcgaaaacgtcgaacgtcatcatcgcttcgccgttgaagccccggcggaacgttacatagcagctggccgcacaaaaaccggctgccagcgaacatacgatgctcagggtcatacgctggggcgaagtcttgctggagttcatgacttcaccgaaatctttgcacccatcgtcaacgcgctccgggtctggcagcaactgcaaacaatatagtacgtatgctttcgcaatcttcgagcaaacgcggttccaatcttttcatgtaaggttggcgtcgcaccatgactgccacgtcctttctgggggtcgcgcgtttcagggcggtcttctgcgtcagcgggcagtcttcggtcggaattgacccttgatatacgtgtactaaggggtacgggtgcgagcgttttgccgagcccgccttgcttgggaacagggactgccctgccgactttggcagtcacatttcttgcgttatttcgcgtctcaaagtcataagcggccggagccaggtcgttgctgtttattagcgataatggcgcgatcgcagaccaatgaagagtagccgccatgtaccgcagaccagtcgacgctcttcccgaggaatcacgcaatatggctgcatttggtaatcttggtgagctagttggaatcagcctatccctaacatctctttgaacattggacagtgcagagctagaggcggatgcggctccacgccgttcgcctgtcagtccgacggaggccgggcgagcagagcgtatcgtgaactgaccatttgccattattcggtccttcctgtctctaggtccccccgcgcccatcgtcgcgacgattacctcttgcacgtttgcgttctcccaattctccatcgtgacggtcacgcggacacgttggagaagcttccggttgctcggtacggatggaagcagacaccggatcagccgccttgttgctgtcatcgcgaccgtcgggctcaccgacatttgcctcggaatcaacttgcgcggccttcaatcttttacggtttgctccgctcggacctgcctcctcgtcatgacgtcgttttgatcgctgctggcgctcgactgtgtgcatggcaacgtcagtcaccgggcgagaagtatcggaattgccgctttgtttgggcacgctggcttcagcgacatacgaagtctcaatcccgaaatgcccagttacagggattttggacgcggatggttgggcaccccgctcgctctccaatggtacgcggatccgtgcgtcggctttgatgctggagccggcgggctcctggaaacgggcgtgcggttcaagcggttgtttgtcatgtcgggttgcacggtccggttcgccggcagatgcgtccgatcgaaatggatcgaacgattgactgaggtcccgacgatcttcctcaggcgaggtctcatcaaaatctgtatcttcgaattgaatcttttgagcctgcatccgctcaaggcgtcgatattcagcatatgtgattggtcgctctgctattcccctttctgctcgcgaagtcgcatccaggactatgccgtgacgaagtgaaatctctgccatctttttccgtaagccgtcataattcagctggggatggcgcctggatattttcagccacccctgccccagaagttcccgacgattgaccacgacatgtaaatgtggatgatcgcggtcgacgtggtaggctgtcagatagttatagcggccacccccgtatcctgacccaaacatctcggctgcccattcccggcttgcttcataagctgcggtttggtcggtacctgcggggaagcttacaataatgtgtgttgttaagtcttgttgcctgtcatcgtctgactgactttcgtcataaatcccggcctccgtaacccagctttgggcaagctcacggatttgatccggcggaacgggaatatcgagatgccgggctgaacgctgcagttccagctttccctttcgggacaggtactccagctgattgattatctgctgaagggtcttggttccacctcctggcacaatgcgaatgattacttgagcgcgatcgggcatccaattttctcccgtcaggtgcgtggtcaagtgctacaaggcacctttcagtaacgagcgaccgtcgatccgtcgccgggatacggacaaaatggagcgcagtagtccatcgagggcggcgaaagcctcgccaaaagcaatacgttcatctcgcacagcctccagatccgatcgagggtcttcggcgtaggcagatagaagcatggatacattgcttgagagtattccgatggactgaagtatggcttccatcttttctcgtgtgtctgcatctatttcgagaaagcccccgatgcggcgcaccgcaacgcgaattgccatactatccgaaagtcccagcaggcgcgcttgataggaaaaggtttcatactcggccgatcgcagacgggcactcacgaccttgaacccttcaactttcagggatcgatgctggttgatggtagtctcactcgacgtggctctggtgtgttttgacatagcttcctccaaagaaagcggaaggtctggatactccagcacgaaatgtgcccgggtagacggatggaagtctagccctgctcaatatgaaatcaacagtacatttacagtcaatactgaatatacttgctacatttgcaattgtcttataacgaatgtgaaataaaaatagtgtaacaacgcttttactcatcgataatcacaaaaacatttatacgaacaaaaatacaaatgcactccggtttcacaggataggcgggatcagaatatgcaacttttgacgttttgttctttcaaagggggtgctggcaaaaccaccgcactcatgggcctttgcgctgctttggcaaatgacggtaaacgagtggccctctttgatgccgacgaaaaccggcctctgacgcgatggagagaaaacgccttacaaagcagtacctgggatcctcgctgtgaagtctattccgccgacgaaatgccccttcttgaagcagcctatgaaaatgccgagctcgaaggatttgattatgcgttggccgatacgcgtggcggctcgagcgagctcaacaacacaatcatcgctagctcaaacctgcttctgatccccaccatgctaacgccgctcgacatcgatgaggcactatctacctaccgctacgtcatcgagctgctgttgagtgaaaatttggcaattcctacagctgttttgcgccaacgcgtcccggtcggccgattgacaacatcgcaacgcaggatgtcagagacgctagagagccttccagttgtaccgtctcccatgcatgaaagagatgcatttgccgcgatgaaagaacgcggcatgttgcatcttacattactaaacacgggaactgatccgacgatgcgcctcatagagaggaatcttcggattgcgatggaggaagtcgtggtcatttcgaaactgatcagcaaaatcttggaggcttgaagatggcaattcgcaagcccgcattgtcggtcggcgaagcacggcggcttgctggtgctcgacccgagatccaccatcccaacccgacacttgttccccagaagctggacctccagcacttgcctgaaaaagccgacgagaaagaccagcaacgtgagcctctcgtcgccgatcacatttacagtcccgatcgacaacttaagctaactgtggatgcccttagtccacctccgtccccgaaaaagctccaggtttttctttcagcgcgaccgcccgcgcctcaagtgtcgaaaacatatgacaacctcgttcggcaatacagtccctcgaagtcgctacaaatgattttaaggcgcgcgttggacgatttcgaaagcatgctggcagatggatcatttcgcgtggccccgaaaagttatccgatcccttcaactacagaaaaatccgttctcgttcagacctcacgcatgttcccggttgcgttgctcgaggtcgctcgaagtcattttgatccgttggggttggagaccgctcgagctttcggccacaagctggctaccgccgcgctcgcgtcattctttgctggagagaagccatcgagcaattggtgaagagggacctatcggaacccctcaccaaatattgagtgtagatttgaggccgctggccgcgtcctcagtcaccttttgagccagataattaagagccaaatgcaattggctcaggctgccatcgtccccccgtgcgaaacctgcacgtccgcgtcaaagaaataaccggcacctcttgctgtttttatcagttgagggcttgacggatccgcctcaagtttgcggcgcagccgcaaaatgagaacatctatactcctgtcgtaaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgcaagaaactctctcgtactaaacggcttagcgataaaatcacttgctcctagctcgagtgcaacaactttatccgtctcctcaaggcggtcgccactgataattatgattggaatatcagactttgccgccagatttcgaacgatctcaagcccatcttcacgacctaaatctagatcaacaaccacgacatcgaccgtcgcggaagagagtactctagtgaactgggtgctgtcggctaccgcggtcactttgaaggcgtggatcgtaaggtattcgataataagatgccgcatagcgacatcgtcatcgataagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacgaaaatgcgaaccaagtatttcaattttatgacaaaagttctcaatcgttgttacaagtgaaacgcttcgaggttacagctactattgattaaggagatcgcctatggtctcgccccggcgtcgtgcgtccgccgcgagccagatctcgcctacttcataaacgtcctcataggcacggaatggaatgatgacatcgatcgccgtagagagcatgtcaatcagtgtgcgatcttccaagctagcaccttgggcgctacttttgacaagggaaaacagtttcttgaatccttggattggattcgcgccgtgtattgttgaaatcgatcccggatgtcccgagacgacttcactcagataagcccatgctgcatcgtcgcgcatctcgccaagcaatatccggtccggccgcatacgcagacttgcttggagcaagtgctcggcgctcacagcacccagcccagcaccgttcttggagtagagtagtctaacatgattatcgtgtggaatgacgagttcgagcgtatcttctatggtgattagcctttcctggggggggatggcgctgatcaaggtcttgctcattgttgtcttgccgcttccggtagggccacatagcaacatcgtcagtcggctgacgacgcatgcgtgcagaaacgcttccaaatccccgttgtcaaaatgctgaaggatagcttcatcatcctgattttggcgtttccttcgtgtctgccactggttccacctcgaagcatcataacgggaggagacttctttaagaccagaaacacgcgagcttggccgtcgaatggtcaagctgacggtgcccgagggaacggtcggcggcagacagatttgtagtcgttcaccaccaggaagttcagtggcgcagagggggttacgtggtccgacatcctgctttctcagcgcgcccgctaaaatagcgatatcttcaagatcatcataagagacgggcaaaggcatcttggtaaaaatgccggcttggcgcacaaatgcctctccaggtcgattgatcgcaatttcttcagtcttcgggtcatcgagccattccaaaatcggcttcagaagaaagcgtagttgcggatccacttccatttacaatgtatcctatctctaagcggaaatttgaattcattaagagcggcggttcctcccccgcgtggcgccgccagtcaggcggagctggtaaacaccaaagaaatcgaggtcccgtgctacgaaaatggaaacggtgtcaccctgattcttcttcagggttggcggtatgttgatggttgccttaagggctgtctcagttgtctgctcaccgttattttgaaagctgttgaagctcatcccgccacccgagctgccggcgtaggtgctagctgcctggaaggcgccttgaacaacactcaagagcatagctccgctaaaacgctgccagaagtggctgtcgaccgagcccggcaatcctgagcgaccgagttcgtccgcgcttggcgatgttaacgagatcatcgcatggtcaggtgtctcggcgcgatcccacaacacaaaaacgcgcccatctccctgttgcaagccacgctgtatttcgccaacaacggtggtgccacgatcaagaagcacgatattgttcgttgttccacgaatatcctgaggcaagacacactttacatagcctgccaaatttgtgtcgattgcggtttgcaagatgcacggaattattgtcccttgcgttaccataaaatcggggtgcggcaagagcgtggcgctgctgggctgcagctcggtgggtttcatacgtatcgacaaatcgttctcgccggacacttcgccattcggcaaggagttgtcgtcacgcttgccttcttgtcttcggcccgtgtcgccctgaatggcgcgtttgctgaccccttgatcgccgctgctatatgcaaaaatcggtgtttcttccggccgtggctcatgccgctccggttcgcccctcggcggtagaggagcagcaggctgaacagcctcttgaaccgctggaggatccggcggcacctcaatcggagctggatgaaatggcttggtgtttgttgcgatcaaagttgacggcgatgcgttctcattcaccttcttttggcgcccacctagccaaatgaggcttaatgataacgcgagaacgacacctccgacgatcaatttctgagaccccgaaagacgccggcgatgtttgtcggagaccagggatccagatgcatcaacctcatgtgccgcttgctgactatcgttattcatcccttcgcccccttcaggacgcgtttcacatcgggcctcaccgtgcccgtttgcggcctttggccaacgggatcgtaagcggtgttccagatacatagtactgtgtggccatccctcagacgccaacctcgggaaaccgaagaaatctcgacatcgctccctttaactgaatagttggcaacagcttccttgccatcaggattgatggtgtagatggagggtatgcgtacattgcccggaaagtggaataccgtcgtaaatccattgtcgaagacttcgagtggcaacagcgaacgatcgccttgggcgacgtagtgccaattactgtccgccgcaccaagggctgtgacaggctgatccaataaattctcagctttccgttgatattgtgcttccgcgtgtagtctgtccacaacagccttctgttgtgcctcccttcgccgagccgccgcatcgtcggcggggtaggcgaattggacgctgtaatagagatcgggctgctctttatcgaggtgggacagagtcttggaacttatactgaaaacataacggcgcatcccggagtcgcttgcggttagcacgattactggctgaggcgtgaggacctggcttgccttgaaaaatagataatttccccgcggtagggctgctagatctttgctatttgaaacggcaaccgctgtcaccgtttcgttcgtggcgaatgttacgaccaaagtagctccaaccgccgtcgagaggcgcaccacttgatcgggattgtaagccaaataacgcatgcgcggatctagcttgcccgccattggagtgtcttcagcctccgcaccagtcgcagcggcaaataaacatgctaaaatgaaaagtgcttttctgatcatggttcgctgtggcctacgtttgaaacggtatcttccgatgtctgataggaggtgacaaccagacctgccgggttggttagtctcaatctgccgggcaagctggtcaccttttcgtagcgaactgtcgcggtccacgtactcaccacaggcattttgccgtcaacgacgagggtccttttatagcgaatttgctgcgtgcttggagttacatcatttgaagcgatgtgctcgacctccaccctgccgcgtttgccaagaatgacttgaggcgaactgggattgggatagttgaagaattgctggtaatcctggcgcactgttggggcactgaagttcgataccaggtcgtaggcgtactgagcggtgtcggcatcataactctcgcgcaggcgaacgtactcccacaatgaggcgttaacgacggcctcctcttgagttgcaggcaatcgcgagacagacacctcgctgtcaacggtgccgtccggccgtatccatagatatacgggcacaagcctgctcaacggcaccattgtggctatagcgaacgcttgagcaacatttcccaaaatcgcgatagctgcgacagctgcaatgagtttggagagacgtcgcgccgatttcgctcgcgcggtttgaaaggcttctacttccttatagtgctcggcaaggctttcgcgcgccactagcatggcatattcaggccccgtcatagcgtccacccgaattgccgagctgaagatctgacggagtaggctgccatcgccccacattcagcgggaagatcgggcctttgcagctcgctaatgtgtcgtttgtctggcagccgctcaaagcgacaactaggcacagcaggcaatacttcatagaattctccattgaggcgaatttttgcgcgacctagcctcgctcaacctgagcgaagcgacggtacaagctgctggcagattgggttgcgccgctccagtaactgcctccaatgttgccggcgatcgccggcaaagcgacaatgagcgcatcccctgtcagaaaaaacatatcgagttcgtaaagaccaatgatcttggccgcggtcgtaccggcgaaggtgattacaccaagcataagggtgagcgcagtcgcttcggttaggatgacgatcgttgccacgaggtttaagaggagaagcaagagaccgtaggtgataagttgcccgatccacttagctgcgatgtcccgcgtgcgatcaaaaatatatccgacgaggatcagaggcccgatcgcgagaagcactttcgtgagaattccaacggcgtcgtaaactccgaaggcagaccagagcgtgccgtaaaggacccactgtgccccttggaaagcaaggatgtcctggtcgttcatcggaccgatttcggatgcgattttctgaaaaacggcctgggtcacggcgaacattgtatccaactgtgccggaacagtctgcagaggcaagccggttacactaaactgctgaacaaagtttgggaccgtcttttcgaagatggaaaccacatagtcttggtagttagcctgcccaacaattagagcaacaacgatggtgaccgtgatcacccgagtgataccgctacgggtatcgacttcgccgcgtatgactaaaataccctgaacaataatccaaagagtgacacaggcgatcaatggcgcactcaccgcctcctggatagtctcaagcatcgagtccaagcctgtcgtgaaggctacatcgaagatcgtatgaatggccgtaaacggcgccggaatcgtgaaattcatcgattggacctgaacttgactggtttgtcgcataatgttggataaaatgagctcgcattcggcgaggatgcgggcggatgaacaaatcgcccagccttaggggagggcaccaaagatgacagcggtcttttgatgctccttgcgttgagcggccgcctcttccgcctcgtgaaggccggcctgcgcggtagtcatcgttaataggcttgtcgcctgtacattttgaatcattgcgtcatggatctgcttgagaagcaaaccattggtcacggttgcctgcatgatattgcgagatcgggaaagctgagcagacgtatcagcattcgccgtcaagcgtttgtccatcgtttccagattgtcagccgcaatgccagcgctgtttgcggaaccggtgatctgcgatcgcaacaggtccgcttcagcatcactacccacgactgcacgatctgtatcgctggtgatcgcacgtgccgtggtcgacattggcattcgcggcgaaaacatttcattgtctaggtccttcgtcgaaggatactgatttttctggttgagcgaagtcagtagtccagtaacgccgtaggccgacgtcaacatcgtaaccatcgctatagtctgagtgagattctccgcagtcgcgagcgcagtcgcgagcgtctcagcctccgttgccgggtcgctaacaacaaactgcgcccgcgcgggctgaatatatagaaagctgcaggtcaaaactgttgcaataagttgcgtcgtcttcatcgtttcctaccttatcaatcttctgcctcgtggtgacgggccatgaattcgctgagccagccagatgagttgccttcttgtgcctcgcgtagtcgagttgcaaagcgcaccgtgttggcacgccccgaaagcacggcgacatattcacgcatatcccgcagatcaaattcgcagatgacgcttccactttctcgtttaagaagaaacttacggctgccgaccgtcatgtcttcacggatcgcctgaaattccttttcggtacatttcagtccatcgacataagccgatcgatctgcggttggtgatggatagaaaatcttcgtcatacattgcgcaaccaagctggctcctagcggcgattccagaacatgctctggttgctgcgttgccagtattagcatcccgttgttttttcgaacggtcaggaggaatttgtcgacgacagtcgaaaatttagggtttaacaaataggcgcgaaactcatcgcagctcatcacaaaacggcggccgtcgatcatggctccaatccgatgcaggagatatgctgcagcgggagcgcatacttcctcgtattcgagaagatgcgtcatgtcgaagccggtaatcgacggatctaactttacttcgtcaacttcgccgtcaaatgcccagccaagcgcatggccccggcaccagcgttggagccgcgctcctgcgccttcggcgggcccatgcaacaaaaattcacgtaaccccgcgattgaacgcatttgtggatcaaacgagagctgacgatggataccacggaccagacggcggttctcttccggagaaatcccaccccgaccatcactctcgatgagagccacgatccattcgcgcagaaaatcgtgtgaggctgctgtgttttctaggccacgcaacggcgccaacccgctgggtgtgcctctgtgaagtgccaaatatgttcctcctgtggcgcgaaccagcaattcgccaccccggtccttgtcaaagaacacgaccgtacctgcacggtcgaccatgctctgttcgagcatggctagaacaaacatcatgagcgtcgtcttacccctcccgataggcccgaatattgccgtcatgccaacatcgtgctcatgcgggatatagtcgaaaggcgttccgccattggtacgaaatcgggcaatcgcgttgccccagtggcctgagctggcgccctctggaaagttttcgaaagagacaaaccctgcgaaattgcgtgaagtgattgcgccagggcgtgtgcgccacttaaaattccccggcaattgggaccaataggccgcttccataccaataccttcttggacaaccacggcacctgcatccgccattcgtgtccgagcccgcgcgcccctgtccccaagactattgagatcgtctgcatagacgcaaaggctcaaatgatgtgagcccataacgaattcgttgctcgcaagtgcgtcctcagcctcggataatttgccgatttgagtcacggctttatcgccggaactcagcatctggctcgatttgaggctaagtttcgcgtgcgcttgcgggcgagtcaggaacgaaaaactctgcgtgagaacaagtggaaaatcgagggatagcagcgcgttgagcatgcccggccgtgtttttgcagggtattcgcgaaacgaatagatggatccaacgtaactgtcttttggcgttctgatctcgagtcctcgcttgccgcaaatgactctgtcggtataaatcgaagcgccgagtgagccgctgacgaccggaaccggtgtgaaccgaccagtcatgatcaaccgtagcgcttcgccaatttcggtgaagagcacaccctgcttctcgcggatgccaagacgatgcaggccatacgctttaagagagccagcgacaacatgccaaagatcttccatgttcctgatctggcccgtgagatcgttttccctttttccgcttagcttggtgaacctcctctttaccttccctaaagccgcctgtgggtagacaatcaacgtaaggaagtgttcattgcggaggagttggccggagagcacgcgctgttcaaaagcttcgttcaggctagcggcgaaaacactacggaagtgtcgcggcgccgatgatggcacgtcggcatgacgtacgaggtgagcatatattgacacatgatcatcagcgatattgcgcaacagcgtgttgaacgcacgacaacgcgcattgcgcatttcagtttcctcaagctcgaatgcaacgccatcaattctcgcaatggtcatgatcgatccgtcttcaagaaggacgatatggtcgctgaggtggccaatataagggagatagatctcaccggatctttcggtcgttccactcgcgccgagcatcacaccattcctctccctcgtgggggaaccctaattggatttgggctaacagtagcgcccccccaaactgcactatcaatgcttcttcccgcggtccgcaaaaatagcaggacgacgctcgccgcattgtagtctcgctccacgatgagccgggctgcaaaccataacggcacgagaacgacttcgtagagcgggttctgaacgataacgatgacaaagccggcgaacatcatgaataaccctgccaatgtcagtggcaccccaagaaacaatgcgggccgtgtggctgcgaggtaaagggtcgattcttccaaacgatcagccatcaactaccgccagtgagcgtttggccgaggaagctcgccccaaacatgataacaatgccgccgacgacgccggcaaccagcccaagcgaagcccgcccgaacatccaggagatcccgatagcgacaatgccgagaacagcgagtgactggccgaacggaccaaggataaacgtgcatatattgttaaccattgtggcggggtcagtgccgccacccgcagattgcgctgcggcgggtccggatgaggaaatgctccatgcaattgcaccgcacaagcttggggcgcagctcgatatcacgcgcatcatcgcattcgagagcgagaggcgatttagatgtaaacggtatctctcaaagcatcgcatcaatgcgcacctccttagtataagtcgaataagacttgattgtcgtctgcggatttgccgttgtcctggtgtggcggtggcggagcgattaaaccgccagcgccatcctcctgcgagcggcgctgatatgacccccaaacatcccacgtctcttcggattttagcgcctcgtgatcgtcttttggaggctcgattaacgcgggcaccagcgattgagcagctgtttcaacttttcgcacgtagccgtttgcaaaaccgccgatgaaattaccggtgttgtaagcggagatcgcccgacgaagcgcaaattgcttctcgtcaatcgtttcgccgcctgcataacgacttttcagcatgtttgcagcggcagataatgatgtgcacgcctggagcgcaccgtcaggtgtcagaccgagcatagaaaaatttcgagagtttatttgcatgaggccaacatccagcgaatgccgtgcatcgagacggcgcctgacgacttgggttgcttggctgtgatcttgccagtgaagcgtttcgccggtcgtgttgtcatgaatcgctaaaggatcaaagcgactctccaccttagctatcgccgcaagcgtagatgtcgcaactgatggggcacacttgcgagcaacatggtcaaactcagcagatgagagtggcgtggcaaggctcgacgaacagaaggagaccatcaaggcaagagaaagcgaccccgatctcttaagcataccttatctccttagctcgcaactaacaccgcctctcccgttggaagaagtgcgttgttttatgttgaagattatcgggagggtcggttactcgaaaattttcaattgcttctttatgatttcaattgaagcgagaaacctcgcccggcgtcttggaacgcaacatggaccgagaaccgcgcatccatgactaagcaaccggatcgacctattcaggccgcagttggtcaggtcaggctcagaacgaaaatgctcggcgaggttacgctgtctgtaaacccattcgatgaacgggaagcttccttccgattgctcttggcaggaatattggcccatgcctgcttgcgctttgcaaatgctcttatcgcgttggtatcatatgccttgtccgccagcagaaacgcactctaagcgattatttgtaaaaatgtttcggtcatgcggcggtcatgggcttgacccgctgtcagcgcaagacggatcggtcaaccgtcggcatcgacaacagcgtgaatcttggtggtcaaaccgccacgggaacgtcccatacagccatcgtcttgatcccgctgtttcccgtcgccgcatgttggtggacgcggacacaggaactgtcaatcatgacgacattctatcgaaagccttggaaatcacactcagaatatgatcccagacgtctgcctcacgccatcgtacaaagcgattgtagcaggttgtacaggaaccgtatcgatcaggaacgtctgcccagggcgggcccgtccggaagcgccacaagatgacattgatcacccgcgtcaacgcgcggcacgcgacgcggcttatttgggaacaaaggactgaacaacagtccattcgaaatcggtgacatcaaagcggggacgggttatcagtggcctccaagtcaagcctcaatgaatcaaaatcagaccgatttgcaaacctgatttatgagtgtgcggcctaaatgatgaaatcgtccttctagatcgcctccgtggtgtagcaacacctcgcagtatcgccgtgctgaccttggccagggaattgactggcaagggtgctttcacatgaccgctcttttggccgcgatagatgatttcgttgctgctttgggcacgtagaaggagagaagtcatatcggagaaattcctcctggcgcgagagcctgctctatcgcgacggcatcccactgtcgggaacagaccggatcattcacgaggcgaaagtcgtcaacacatgcgttataggcatcttcccttgaaggatgatcttgttgctgccaatctggaggtgcggcagccgcaggcagatgcgatctcagcgcaacttgcggcaaaacatctcactcacctgaaaaccactagcgagtctcgcgatcagacgaaggccttttacttaacgacacaatatccgatgtctgcatcacaggcgtcgctatcccagtcaatactaaagcggtgcaggaactaaagattactgatgacttaggcgtgccacgaggcctgagacgacgcgcgtagacagttttttgaaatcattatcaaagtgatggcctccgctgaagcctatcacctctgcgccggtctgtcggagagatgggcaagcattattacggtcttcgcgcccgtacatgcattggacgattgcagggtcaatggatctgagatcatccagaggattgccgcccttaccttccgtttcgagttggagccagcccctaaatgagacgacatagtcgacttgatgtgacaatgccaagagagagatttgcttaacccgatttttttgctcaagcgtaagcctattgaagcttgccggcatgacgtccgcgccgaaagaatatcctacaagtaaaacattctgcacaccgaaatgcttggtgtagacatcgattatgtgaccaagatccttagcagtttcgcttggggaccgctccgaccagaaataccgaagtgaactgacgccaatgacaggaatcccttccgtctgcagataggtaccaatcgcctcatcgatattttgccatccagcgtctccggaataaatgatcgccaccgtgttcttggctggcttggcattgaacacccgaagaggaagcctcactccagtttcgcccccgtttgaccacatgaaaccgttggcgcggtcattcgcatatgcagcgtctgccgcgggaaagagtgtgaatacgagtatcaataccaattttatcgccatttcgataccttatcagcggagatctcgataatttcggactaatgtttcaagtggatatcaactgcgtcgcaatttgttacaaaagacacgtaagcccgcggtgaaggtttgcggagaacttttgctgagatctcgaatcgaaagaagcacttaaagttctgcaacaagcgcgcctgatgcacgtcaccacgctgatgacggaatgggcgatattcctgatggggtggctctgtttaccacgtaagcggcaagctgaggccgtaagacccgggtataaagggcgaattccagcacactggcggccgttaggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagaccatttaaatcctaggcctgcaggaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgaactagtctgtacccgatcaacaccgagacccgtggcgtcttcgacctcaatggcgtctggaacttcaagctggactacgggaaaggactggaagagaagtggtacgaaagcaagctgaccgacactattagtatggccgtcccaagcagttacaatgacattggcgtgaccaaggaaatccgcaaccatatcggatatgtctggtacgaacgtgagttcacggtgccggcctatctgaaggatcagcgtatcgtgctccgcttcggctctgcaactcacaaagcaattgtctatgtcaatggtgagctggtcgtggagcacaagggcggattcctgccattcgaagcggaaatcaacaactcgctgcgtgatggcatgaatcgcgtcaccgtcgccgtggacaacatcctcgacgatagcaccctcccggtggggctgtacagcgagcgccacgaagagggcctcggaaaagtcattcgtaacaagccgaacttcgacttcttcaactatgcaggcctgcaccgtccggtgaaaatctacacgaccccgtttacgtacgtcgaggacatctcggttgtgaccgacttcaatggcccaaccgggactgtgacctatacggtggactttcaaggcaaagccgagaccgtgaaagtgtcggtcgtggatgaggaaggcaaagtggtcgcaagcaccgagggcctgagcggtaacgtggagattccgaatgtcatcctctgggaaccactgaacacgtatctctaccagatcaaagtggaactggtgaacgacggactgaccatcgatgtctatgaagagccgttcggcgtgcggaccgtggaagtcaacgacggcaagttcctcatcaacaacaaaccgttctacttcaagggctttggcaaacatgaggacactcctatcaacggccgtggctttaacgaagcgagcaatgtgatggatttcaatatcctcaaatggatcggcgccaacagcttccggaccgcacactatccgtactctgaagagttgatgcgtcttgcggatcgcgagggtctggtcgtgatcgacgagactccggcagttggcgtgcacctcaacttcatggccaccacgggactcggcgaaggcagcgagcgcgtcagtacctgggagaagattcggacgtttgagcaccatcaagacgttctccgtgaactggtgtctcgtgacaagaaccatccaagcgtcgtgatgtggagcatcgccaacgaggcggcgactgaggaagagggcgcgtacgagtacttcaagccgttggtggagctgaccaaggaactcgacccacagaagcgtccggtcacgatcgtgctgtttgtgatggctaccccggagacggacaaagtcgccgaactgattgacgtcatcgcgctcaatcgctataacggatggtacttcgatggcggtgatctcgaagcggccaaagtccatctccgccaggaatttcacgcgtggaacaagcgttgcccaggaaagccgatcatgatcactgagtacggcgcagacaccgttgcgggctttcacgacattgatccagtgatgttcaccgaggaatatcaagtcgagtactaccaggcgaaccacgtcgtgttcgatgagtttgagaacttcgtgggtgagcaagcgtggaacttcgcggacttcgcgacctctcagggcgtgatgcgcgtccaaggaaacaagaagggcgtgttcactcgtgaccgcaagccgaagctcgccgcgcacgtctttcgcgagcgctggaccaacattccagatttcggctacaagaacgctagccatcaccatcaccatcacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcc

(19) United States
(12) Patent Application Publication
(10) Pub. No.: US 2009/0075358 A1
(43) Pub. Date: Mar. 19, 2009

(54) VECTORS FOR TRANSFORMATION OF PLANT CELLS

(75) Inventors: OSMAT AZZAM JEFFERSON, GOOGONG (AU); RICHARD ANTHONY JEFFERSON, GOOGONG (AU)

Correspondence Address: CAROL NOTTENBURG, 814 32ND AVE 5, SEATTLE, WA, 98144, US

(73) Assignee: CAMBIA, CANBERRA (AU)

(21) Appl. No.: 12/058,704

(22) Filed: Mar. 29, 2008

Related U.S. Application Data
(60) Provisional application No. 60/908,928, filed on Mar. 29, 2007.

(51) Int. Cl. C12N 1/21; C12N 15/00

(52) U.S. Cl. 435/252.3; 435/320.1

(57) ABSTRACT

Vectors for transformation of plants contain a minimum set of transfer machinery genes, such as vir genes from Agrobacterium Ti plasmids or tra genes.

### CROSS-RELATED APPLICATIONS

This patent application claims priority from U.S. Provisional Patent Application No. 60/908,928, filed 29 Mar. 2007.

### TECHNICAL FIELD

This patent application relates generally to technologies for the transfer of nucleic acids molecules to eukaryotic cells and in particular technologies using unitary, binary or cointegrate vectors to transfer nucleic acid sequences to eukaryotic cells, e.g. to plant cells, that have a Type IV secretion system.

### BACKGROUND

Use of transformation technology in plants generally follows three steps: (i) introduction of new DNA into appropriate plant cells/organs; (ii) growth or multiplication of successfully transformed cells/plants, often involving selection or discrimination methodologies; and (iii) expression of transgene(s) in target cells/organs/stages.

Each of these processes can be affected by several alternative technologies of varying quality and efficiencies. For transformation of plant cells, typically one of two types of vectors is used: binary vectors and co-integrate vectors.

The basic elements of the vectors designed for Agrobacterium-mediated transformation were based on native Ti-plasmids; these elements include T-DNA border sequences, at least the right border, which initiates the integration of the T-DNA region into the plant genome, vir genes, which are required for transfer of the T-DNA region to the plant, and a modified T-DNA region of the Ti plasmid, in which the genes responsible for tumor formation are removed by genetic engineering and replaced by foreign genes of diverse origin, e.g., from plants, bacteria, virus. When these oncogenes are removed, transformed plant tissues or cells regenerate into normal-appearing plants and, in most cases, fertile plants.

In binary vector systems, the T-DNA and the vir region reside in separate plasmids within the same Agrobacterium strain. The vir genes are located in a disarmed (without tumor genes) Ti plasmid and the T-DNA linked to one or more genes of interest is located in a small vector molecule.

In co-integrated vectors, the vector is the result of recombination of a small vector plasmid, for example an E. coli vector, and a Ti plasmid harbored in A. tumefaciens. The recombination takes place through a homologous region present in both of the plasmids. An engineered T-DNA containing the gene of interest can initially be located in either one of the plasmids. For a variety of reasons, co-integrated vectors are generally popular to use.

### SUMMARY

There are claims directed to a vector for transformation of plant cells, comprising: (a) virB, virC, virD, and virE operons; (b) at least one oriT or T-DNA border sequence, operatively linked to (c) a sequence of interest. The vector may also include virA and virG operons or a virJ operon or all three operons. The virG protein may be a mutant that functions independent of a virA product. In any of these configurations, the vir operons can be controlled by a heterologous promoter(s). While not necessary, the promoter can be inducible. In some implementations, the vector has an origin of replication that is functional in at least one non-Rhizobiales species. The vector may be a plasmid or integrated into the bacterial chromosome. Exemplary plasmids include pCAMBIA5105 and pCAMBIA5106.

Claims are also directed to a vector system for transformation of plant cells, comprising (a) a first vector comprising at least one oriT or T-DNA border sequence, operatively linked to a sequence of interest; and (b) a second vector comprising a set of vir operons, wherein the set consists of virB, virC, virD, and virE operons. The first and second vectors may further comprise an origin of replication that is functional in at least one non-Rhizobiales species. Optionally, the second vector may further comprise a virA operon and a virG operon or a virJ operon or all three. The vir coding regions may be controlled by one or more heterologous promoters. As above, the virG product can be a mutant. The vectors can be plasmids or the second vector is integrated into a bacterial host chromosome.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entirety.

### BRIEF DESCRIPTION OF THE DRAWINGS

FIG. 1 is a schematic of pCAMBIA5105 vector.

FIGS. 2A and 2B show GUS expression patterns as observed in regenerated tobacco shoots that were transformed with (a) LBA288 (pCAMBIA5105) or (b) Sme (pCAMBIA5105) and stained for GUS.

FIG. 3 shows GUS expression patterns of regenerated rice shoots that were transformed with Sme (pCAMBIA5105) and stained for GUS.

FIGS. 4A and 4B present results of amplification for signature fragments of pCAMBIA5105 and pCAMBIA5106 in various bacteria strains. FIG. 4A assays for pCAMBIA5105 using (A) virD4 Fwd and Rev primers; (D) virAFw and Rev primers; (E) virE3 Fw and Rev primers; (H) virJ Fw and Rev primers; or (I) TopoKmRFw and Rev primers; or for signature fragments of bacterial genes using (B) NGR234Nod D1Fwd and Rev primers; (C) Mlo16SrRNAFwd and Rev primers; (F) Rle16SrRNAFw and Rev primers; or (G) or RtrNodDFw and Rev primers (see Table 3). Unless stated, the numbers in each of the frames represent individually screened colonies. The numbers 1, 2, and 3 in results for R. leguminosarum bv trifolii strain ANU843 refer to ANU843 (pCAMBIA5105), ANU843 WT, and ANU845WT correspondingly. M refers to 1 kb marker, (−) negative control or distilled water, (p) pCAMBIA5105 purified DNA, and (+) a bacterial colony known to contain pCAMBIA5105. FIG. 4B shows amplifications assays for pCAMBIA5106. Two colonies were checked with the following primer sets: (A) Mlo16SrRNA Fw and Rev (specific to M. loti), (B) TopoKmRFw and Rev, (C) MloinsFw and Rev (specific to M. loti R7A strain), (D) virE3 Fw and Rev, (E) Hyg510 Fw and Rev, (S) specific primers for Sinorhizobium strain, (X) negative control primer sets for M loti, and (Y) negative control primer set for Sinorhizobium. M refers to 1 kb marker.

FIG. 5 shows transformation of tobacco seedlings with LBA288(pCAMBIA5105), ANU845(pCAMBIA5105), and Sme (pCAMBIA5105).

FIGS. 6A and B are close ups of transformed tobacco seedlings (A) and rice seedlings/calli (B) stained for GUS.

FIGS. 7A and 7B Amplification results (a) for Pseudomonas (pCAMBIA5105) colonies 6 and 25. Five sets of primers were used: (1) Pseud16SFW&Rev; (2) TopoKmRFw&Rev; (3) Spec-SacII Fwd&Rev; (4) SpecIntFwd &topoKmRev; and (5) HygFwd&virA seq rev. The numbers represent the primer set used. M refers to 1 kb marker, (−) negative control or distilled water, (p) pCAMBIA5105 purified DNA, and (+) a wild type bacterial colony. GUS foci (b) as observed on tobacco seedlings 7 days post co-cultivation.

FIGS. 8A and 8B present maps for pOAJG73.2 and pOAJJ66.1 (A) and pOAJJ67.6 (B)

FIG. 9 shows GUS expression at 4 (LBA288) or 7 days post transformation in tobacco seedlings transformed with either LBA288 or ANU845 that contained various constructs.

FIGS. 10A and 10B are close up photographs of GUS-assayed tobacco seedlings (A) and rice seedlings/calli (B).

FIG. 11 is a drawing of the PCR2.1Rc-based cloning vector containing lacIQ and p15A origin of replication (pOAJH111).

FIGS. 12A and 12B show a strategy for constructing a lacpWT vir regulon and GT-bacK-Pac vector.

FIG. 13 is a drawing of pCAMBIA5106.

FIG. 14 shows transformation of tobacco seedlings with pCAMBIA5106, a ΔvirJ construct, or lacpvirE construct.

### DETAILED DESCRIPTION

The present disclosure provides vectors for transformation that have the minimum necessary genes for transfer of the vector (e.g., vir genes) and at least one oriT or T-DNA border sequence, which is operatively linked to a sequence of interest. Some of the vectors have a wide host range and can replicate in at least one bacterial species other than Agrobacterium or, in some circumstances, other than those in the order Rhizobiales. Furthermore, the vectors can be either a single (“unitary”) vector, which does not require a helper vector for transformation, or a “binary” vector system, which does require a helper vector.

In one vector system, a single vector comprises genes encoding molecules required for transfer to eukaryotic cells as well as a gene or nucleic acid sequence of interest operatively linked to T-DNA sequences or oriT. This single vector is called herein a “unitary” vector. As discussed above, the unitary vector comprises a minimum set of transfer genes, typically vir genes and typically vir genes from Ti plasmids found in Agrobacterium.

In another implementation of a transfer vector system, two vectors are employed. Such a system is herein called a “binary” vector system. In a binary vector system, there are: (i) a vector, which is often a “disarmed” Ti plasmid, meaning that it is not tumorigenic because its tumor-inducing genes located in the T-DNA have been removed, and (ii) a wide-host-range vector. As used herein, “wide-host-range” means that the vector replicates in at least one bacterial species other than, or in addition to, Agrobacterium. Host range is conferred by an origin of replication. Typical ori that could be used permit the replication or maintenance of the plasmid in a wide range of bacteria. (U.S. Pat. Nos. 4,940,838; 5,149,645; 6,165,780; 6,265,638).

Although transforming vector(s) are generally described herein as plasmids, the vector systems can be nucleic acid molecules other than plasmids. For example, vectors can be integrated into a bacterial genome. For integrated vectors however, the starting material is generally a plasmid.

The following description applies to a binary vector system and a unitary vector system as well as a vector system in which one or two vectors are integrated into a host bacteria genome. For a vector that is integrated, it will be readily recognized that some of the components (e.g., origin of replication) are optional. Because the components apply to any of the systems, the discussion is neutral with respect to the actual system employed.

### A. Transfer Genes

Either type of vector, unitary or binary, contains the minimum set of genes required to transfer DNA. Typically, the transfer genes are vir genes of a Ti plasmid. The minimum set contains a virB operon, a virC operon, a virD operon, and a vire operon (see Komari et al. J Bacteriol 166: 88-94, 1986; Schrammeijer et al. J Exp Botany 51: 1167-1169, 2000). The virB, virC, virD, and virE operons each comprise a plurality of structural genes. For example, one of the vectors of the Examples has virB1, virB2, virB3, virB4, virB5, virB6, virB7, virB8, virB9, virB10, virB11, and virB12; virC1, virC2; virD1, virD2, virD3, virD4, virD5; vire1, virE2, and virE3. These vir genes were isolated from pTiBo542. Other Ti plasmids, such as pTi15955 (GenBank Accession No; AF242881), pTiC58 (GenBank Accession No: NC 00492), pTi-SAKURA (Hattori et al. Genes Genet Syst 76: 121-130, 2001); and pTiA6, can be used as starting material for isolation of the vir genes.

In some vectors, additional vir genes are included. For example, a vector may also have virA and virG or virG—especially one of the virG mutants described below—or a vector may also have virJ, whether or not it has virA+virG or virG. VirJ is believed to be functionally redundant with chromosomal AcvB. For host bacterial strains that have acvB, virJ is typically not included in the vector. The

In other vectors, virA and virG genes are present or a mutant virG gene is present. VirG is a transcriptional activator that is regulated by VirA, which functions as an environmental sensor. Several different mutations in virG however, render the activity of the VirG protein independent of VirA. In particular, the single mutant N54D and the triple mutant V50A, N54D, G56A are able to interact with RNA polymerases in bacterial species other than Agrobacterium. The mutations of virG can be generated by any of a number of methods, including site-directed mutagenesis and replacement of the region with a synthetically made nucleic acid.

Another way to regulate vir gene expression is to replace the native promoter region sequences of the vir genes with heterologous promoters. When the native vir gene promoters are replaced by heterologous promoters, the gene products of virG and virA are not necessary for expression of the vir genes and are typically not found in the vectors.

The heterologous promoters can be any promoter that is functional in the host bacteria. It can be a prokaryotic promoter, a viral or bacteriophage promoter, a eukaryotic promoter, or synthetic promoter. Most often, the promoter will be from a prokaryote, virus or bacteriophage. While the promoter can be constitutively active, more often the promoter will be inducible. When the heterologous promoter(s) are inducible by exogenously applied materials or by endogenously expressed products, the gene transfer machinery can be regulated in a purposeful manner. The heterologous promoter needs to be active in the host bacterial cell so that the vir genes are expressed. Examples of heterologous inducible promoters include tac, a hybrid of E. coli tac and lac promoters, tet promoter, lac promoter, araBAD promoter, 1-rhamnose inducible rhaP BAD promoter, among others. Inducible promoters may respond to biological or physical factors. These factors include presence (or absence) of specific sugars, presence of ethanol, starvation for nutrients or oxygen, temperature (e.g., heat shock), abnormal salt concentration, superoxides, abnormal pH, etc. Sequences of inducible promoters are readily available from GenBank, various genome sequence projects (which can be centrally accessed through NCBI), the Genomes OnLine Database (currently version 2.0) (Liolios et al., Nucl. Acids Res. 34 (Database issue):D332-334, 2006), and so on. Interchange of the native vir promoters with the heterologous promoters can be accomplished by a variety of methods, including simple restriction fragment substitution or by subcloning individual vir operons and amplifying the operons using primers having adapter sequences at their ends. In this case, the heterologous promoter has complementary adapter sequences, and can be ligated to the operons. The operons with substituted promoters are then moved into a vector backbone.

### B. Mobilization Sequences and Nucleic Acid Sequence of Interest

The nucleic acid sequence(s) of interest are operatively linked with sequences required in the mobilization process to the target eukaryotic cell. In many vectors, the left and right T-DNA borders (or at least the right T-border) flank the sequence of interest. In maps, the borders are usually designated as LB (left border) and RB (right border). The T-DNA border sequences are 25 bp in length and highly homologous in sequence. In a Ti plasmid, the border sequences flank the T-region in a directly repeated orientation. The sequences are the target of VirD1/VirD2 endonuclease that is involved in releasing the sequence located between the T-DNA borders (Gelvin, Microbiol. and Mol. Biol. Rev. 67:16-37, 2003). While many vectors incorporate both the left and right T-DNA borders, the right T-DNA border is sufficient. When two border sequences are used, the DNA sequence of interest is located in between the border sequences. When only one border is used, the sequence of interest is located close enough to the border sequence and in a position to be transferred into the target eukaryotic cells. In certain vectors, the sequence of interest is instead flanked by origin of transfer (oriT) sequences or other sequence that is capable of forming a relaxosome (US 2003/0087439A1, incorporated in its entirety). This type of transfer origin allows the mobilization of the Ti plasmid to other bacteria, e.g. to rhizobia, with the help of the transfer functions of RK2/RP4 or similar vectors, including derivatives. An exemplary mobilizable plasmid is derived from RSF1010 (Scholz et al., Gene 75 (2), 271-288, 1989, GenBank Accession M28829) and CloDF13 (Escudero et al., Mol. Microbiol. 47:891-901, 2003; GenBank Accession NC002119). Other specific oriTs, such as the oriT of RK2/RP4 can also be used (Stabb and Ruby, Enzymol. 358:413-426, 2002).

The sequence of interest, although often a gene sequence, can actually be any nucleic acid sequence whether or not it produces a protein, an RNA, an antisense molecule or regulatory sequence or the like. Sequences of interest (herein called “transgenes”) for introduction into plants may encode proteins that affect fertility, including male sterility, female fecundity, and apomixis; plant protection genes, including proteins that confer resistance to diseases, bacteria, fungus, nematodes, herbicides, viruses and insects; genes and proteins that affect developmental processes or confer new phenotypes, such as genes that control meristem development, timing of flowering, cell division or senescence (e.g., telomerase), toxicity (e.g., diphtheria toxin, saporin), affect membrane permeability (e.g., glucuronide permease (U.S. Pat. No. 5,432,081)), transcriptional activators or repressors, alter nutritional quality, produce vaccines, and the like. Insect and disease resistance genes are well known. Some of these genes are present in the genome of plants and have been genetically identified. Others of these genes have been found in bacteria and are used to confer resistance. Particularly well known insect resistance genes are the genes encoding the crystal proteins of Bacillus thuringiensis. The crystal proteins are active against various insects, such as lepidopterans, Diptera, Hemiptera and Coleoptera. Many of these genes have been cloned. For examples, see, GenBank; U.S. Pat. Nos. 5,317,096; 5,254,799; 5,460,963; 5,308,760, 5,466,597, 5,187,091, 5,382,429, 5,164,180, 5,206,166, 5,407,825, 4,918,066. Other resistance genes to Sclerotinia, cyst nematodes, tobacco mosaic virus, flax and crown rust, rice blast, powdery mildew, verticillum wilt, potato beetle, aphids, as well as other infections, are useful within the context of this invention. Nucleotide sequences for other transgenes, such as controlling male fertility, are found in U.S. Pat. No. 5,478,369, references therein, and Mariani et al., Nature 347:737, 1990.

Other transgenes that are useful for transforming plants include sequences to make edible vaccines (e.g. United States Patent No: U.S. Pat. No. 6,136,320; U.S. Pat. No. 6,395,964) for humans or animals, alter fatty acid content, change amino acid composition of food crops (e.g. U.S. Pat. No. 6,664,445), introduce enzymes in pathways to synthesize vitamins such as vitamin A and vitamin E, increase iron concentration, control fruit ripening, reduce allergenic properties of e.g., wheat and nuts, absorb and store toxic and hazardous substances to assist in cleanup of contaminated soils, alter fiber content of woods, increase salt tolerance and drought resistance, amongst others.

In some applications, the transgene is transcribed into antisense RNA. Antisense RNA has been used in transgenic plants to confer resistance to virus (e.g., U.S. Pat. No. 5,316,930), control production of fertile pollen resulting in male-sterile plants (U.S. Pat. No. 5,356,799 and family members), reduce lignin content (e.g., U.S. Pat. No. 6,066,780), alter starch production (e.g., U.S. Pat. No. 5,792,920), affect other synthetic and metabolic pathways, and the like. In general, the antisense RNA is complementary to a plant gene sequence, and upon transcription of the antisense and plant gene sequences, they anneal, which inhibits translation of the plant gene.

In a typical vector, the sequence of interest is operatively linked to a promoter. The product of the sequence of interest may be expressed constitutively, after induction, in selective tissues or at certain stages of development. Regulatory elements to effect such expression are well known in the art. Many examples of regulatory elements may be found in the Patent Lens document “Promoters used to regulate gene expression” version 1.0, October 2003 (incorporated in its entirety). Other promoters can be identified through a variety of assays designed to uncover promoters. Enhancer elements or other regulatory elements can be included in addition to a promoter. “Minimal promoter” sequences, such as the so-called minimal 35S promoter from cauliflower mosaic virus, usually require an enhancer element for activity.

### C. Origins of Replication

When the vector is a plasmid, it will typically contain an origin of replication (ori) functional in the host bacterium. When the ori has a “broad host range”, it is functional in at least two different bacterial species. If bacterial species and genera other than Agrobacterium are used for plant transformation (U.S. Application No. 60/908,928, incorporated in its entirety), then the ori should be functional in those bacteria. Some Ti plasmids found in Agrobacterium are capable of replicating in other genera of bacteria, such as rhizobia. Exemplary rhizobia include Rhizobium leguminosarum bv. trifolii (former R. trifolii), Rhizobium spp. NGR234, Mesorhizobium loti, Phyllobacterium myrsinacearum, and Sinorhizobium meliloti (former R. meliloti), all of which are capable of supporting and expressing the genes of a Ti plasmid. Alternatively, the vector that comprises the transfer genes, such as vir genes of the Ti plasmid or homologous genes, such as tra genes, is a broad-host range plasmid so that it can replicate in bacteria in addition to those listed above. The origin of replication from a broad-host range plasmid, such as pVS1, is used. The vector may also contain other origins of replication, e.g., pBR322 ori, a colE1 type origin or f1 origin. When the nucleic acid molecule is integrated into the bacterial chromosome or other self-replicating bacterial DNA molecule, an origin is not necessary. Thus, when suitably modified and engineered, these bacteria may be used for transferring nucleic acid sequences into eukaryotic cells, and especially into plant cells.

### D. Other Elements

Generally, the vector—be it a component of a binary system or a unitary vector—containing the sequence of interest also contains a selectable or a screenable marker for identifying transformants. The marker preferably confers a growth advantage under appropriate conditions. Well known selectable markers are drug resistance genes, such as neomycin phosphotransferase, hygromycin phosphotransferase, herbicide resistance genes, and the like. Other selection systems, including genes encoding resistance to other toxic compounds, genes encoding products required for growth of the cells, such as in positive selection, can alternatively be used. Examples of these “positive selection” systems are abundant (see for example, U.S. Pat. No. 5,994,629). Alternatively, a screenable marker may be employed that allows the selection of transformed cells based on a visual phenotype, e.g. β-glucuronidase or green fluorescent protein (GFP) expression. The selectable marker also typically has operably linked regulatory elements necessary for transcription of the genes, e.g., constitutive or inducible promoter and a termination sequence, including a polyadenylation signal sequence. Elements that enhance efficiency of transcription are optionally included.

Exemplary small replicon vectors suitable for use in the present invention are based on pCAMBIA 1105.1 or pCAMBIA1305.2. Other vectors have been described (U.S. Pat. Nos. 4,536,475; 5,733,744; 4,940,838; 5,464,763; 5,501,967; 5,731,179) or may be constructed based on the guidelines presented herein. The pCAMBIA 5105 plasmids contain a left and right border sequence for integration into a plant host chromosome and also contain a bacterial origin of replication and selectable marker. These border sequences flank two genes. One is a hygromycin resistance gene (hygromycin phosphotransferase or HYG) driven by a double CaMV 35S promoter and using a nopaline synthase polyadenylation site. The second is the β-glucuronidase (GUS) gene (reporter gene) from any of a variety of organisms, such as E. coli, Staphyloccocus, Thermatoga maritima and the like, under control of the CaMV 35S promoter and nopaline synthase polyadenylation site. If appropriate, the CaMV 35S promoter is replaced by a different promoter.

To assist construction of bacterial strains that have a binary vector system, the Ti plasmid may contain a selectable marker, compatible origins of replication, and multiple virG sequences. Although the selectable marker can be the same on both plasmids, preferably the markers are different so as to facilitate confirmation that both plasmids are present. The transfer plasmid (small replicon or mobilizable vector) can optionally contain at least one additional wild-type or modified virG gene. The additional virG gene(s) can be inserted into the Ti plasmid by any of a variety of methods, including the use of transposons and homologous recombination (Kalogeraki and Winans, Gene 188:69-75, 1997). Homologous recombination can be induced by any method, including the use of a suicide plasmid carrying a cloned fragment of the Ti plasmid (e.g. the virG gene), or a stable replicon that is forced to recombine with the Ti plasmid, e.g. by incompatibility. In addition a gene encoding antibiotic resistance can be included.

An exemplary helper plasmid is pTiBo542. This highly virulent plasmid is also completely sequenced (P. Oger, unpublished data; but see GenBank Accession No. AF242881). Disarmed derivatives pEHA11 and pEHA105 have been widely used (Hood et al., J. Bacteriol. 168:1291-1301, 1986; Hood et al., Transgenic Research 2:208-218, 1993). Other helper plasmids include those of LBA4404, the pGA series, pCG series and others (see, Hellens and Mullineaux, A guide to Agrobacterium binary Ti-vectors. Trends Plant Sci. 5: 446-451, 2000).

### E. Transfection of Bacteria

In general, the plasmids are transferred via conjugation or through a direct transfer method to the bacteria of this invention. By transferring either binary or unitary vectors as described herein, transformation competent bacteria are generated. These bacteria can be used to transform plants and plant cells.

Bacterial species that can take up the vector or vectors and transfer sequences of interest to plant cells are bacteria that have a Type four secretion (T4S) system. Conjugation machinery is an example of a T4S system. T4S is capable of transferring large nucleoprotein conjugation intermediates and target a diversity of cells—bacteria, fungal cells, plant cells, and animal cells—for delivery. Many different bacteria have a T4S system, including both pathogenic and non-pathogenic bacteria (Christie and Vogel, Trends in Microbiol. 8: 354-360, 2000). A number of these bacteria are known to interact with plants in specific manner—e.g., Rhizobium, while others may interact with different types of plant cells or through different mechanisms. The Agrobacterium transfer system is considered a classic T4S system.

Plasmids can be transferred by biological methods, such as conjugation, from Agrobacterium (or other rhizobia), or by physical methods, such as electroporation or mediated by PEG (polyethylene glycol). When transferring plasmids from Agrobacterium tumefaciens to a chosen bacterial strain, the procedure is aided if Agrobacterium has a chromosomal negative selection marker(s), such as auxotrophy or antibiotic sensitivity. Constitutive conjugation ability of the Ti plasmid can be achieved by deletion of accR and/or traM genes on the plasmid (Teyssier-Cuvelle et al., Molec. Ecol. 8:1273-1284, 1999). Otherwise, induction of conjugation can be achieved by use of specific opines, naturally produced in crown galls, or utilizing a self-transmissible R plasmid (e.g. R772 or RP4) which may (temporarily) form a co-integrate with the Ti-based plasmid. If the Ti-based plasmid has been engineered by insertion of a foreign oriT, e.g. the oriT of RP4/RK2, then conjugation from one bacterium to another bacterium can be achieved with the help of bacterial strains, e.g. E. coli, containing compatible transfer functions on a plasmid or on their chromosomes. This may be done in a triparental mating between donor, acceptor and helper strain, or in a biparental mating between a donor containing the transfer genes and an acceptor. Bacteria are transferred to selective medium and putative transconjugants are plated out to isolate single cell colonies. Following transconjugation, the Agrobacterium may be selected against. If the Agrobacterium is sensitive to an antibiotic that the recipient bacteria are resistant to, either naturally resistant or resistant as a result of having the small replicon plasmid, then that antibiotic may be used to select for the recipient bacterial strain. Similarly, if a helper strain was used, it may be selected against by using the same or a different antibiotic to which the recipient bacteria are resistant. They may also be made antibiotic resistant by integration of a foreign gene conferring antibiotic resistance, e.g. mediated by a transposon vector. Similarly, bacteria that have not taken up the Ti plasmid may be eliminated by selection for the Ti plasmid. Generally this selection will be an antibiotic selection as well, but will depend on the selectable markers in the Ti plasmid.

The presence of the unitary or Ti-based plasmid can be verified by any suitable method, although for ease, amplification of the vir genes or any other Ti plasmid sequence is commonly employed. Vir gene expression in the new host can be checked after induction with acetosyringone using any of a variety of assays, such as Northern blotting, RT-PCR, real-time amplification, hybridization on microarrays, Western blots, analysis of gene expression from a reporter gene linked to the promoter of a vir gene and the like.

Plasmids may also be transferred to other bacteria without the use of Agrobacterium as a donor strain. For example, a rhizobial strain that has acquired the plasmid by one or another means may act as the donor of the plasmid to other bacterial acceptor strains. This may in some cases avoid the interference of restriction endonuclease systems that exist in many if not all bacteria.

Instead of conjugation, the plasmid may be electroporated into the recipient bacteria. Isolation of the plasmid and electroporation to other Agrobacterium strains, e.g. to the Ti plasmid cured strain LBA288, has been described (Mozo et al., Plant Mol. Biol. 16:617-918, 1990). Similarly, electroporation may be performed to other bacterial species.

Eukaryotic cells may be transformed within the context of this invention. Moreover, either individual cells or aggregations of cells, such as organs or tissues or parts of organs or tissues may be used. Generally, the cells or tissues to be transformed are cultured before transformation, or cells or tissues may be transformed in situ. In some embodiments, the cells or tissues are cultured in the presence of additives to render them more susceptible to transformation. In other embodiments, the cells or tissues are excised from an organism and transformed without prior culturing.

Suitable eukaryotic organisms as sources for cells or tissues to be transformed include plants, fungi, and yeast. Yeast cells can be transformed with Agrobacterium and so can be used in the context of this invention to measure efficiency of transformation and for optimization of conditions. An advantage of using yeast is their fast growth rate and the ability to grow it in laboratory conditions. Transformants can be easily detected by their changed phenotype, e.g. growth on a medium lacking an essential growth component on which the untransformed cells cannot grow. Quantization of transformation efficiency is then achieved by counting the number of colonies growing on this selective medium. Yeast cell transformation by Agrobacterium occurs independent of the expression of attachment genes necessary for plant transformation, and, by the use of autonomously replicating DNA units (mini-chromosomes), can avoid the need for gene integration if desired. The uncoupling of attachment and DNA integration from the overall gene transfer processes may simplify the optimization of transformation by other bacteria. For example, following Ti/T-DNA plasmid transfer to these bacteria, the system may be optimized by genetic complementation using an A. tumefaciens genomic library transferred to the pTi-bearing bacteria. The bacterial library is then used to transform yeast cells and the bacterial clones that transform most efficiently are selected.

Alternatively, as Agrobacterium tumefaciens and some of the bacterial species have been fully sequenced and can be compared, missing genes in the latter bacteria that are important for transformation by Agrobacterium may be individually picked from the Agrobacterium genome and inserted into the bacterial genome by any means or expressed on a plasmid. Similarly, the bacteria can be used to transform yeast cells under a variety of test conditions, such as temperature, pH, nutrient additives and the like. The best conditions can be quickly determined and then tested in transformation of plant cells or other eukaryotic cells.

Briefly, in an exemplary transformation protocol, plant cells are transformed by co-cultivation of a culture of bacteria containing the unitary or binary vectors described herein with leaf disks, protoplasts, meristematic tissue, shoots or calli to generate transformed plants (Bevan, Nucl. Acids. Res. 12:8711, 1984; U.S. Pat. No. 5,591,616). After co-cultivation for a few days, bacteria are removed, for example by washing and treatment with antibiotics, and plant cells are transferred to post-cultivation medium plates generally containing an antibiotic to inhibit or kill bacterial growth (e.g., cefotaxime) and optionally a selective agent, such as described in U.S. Pat. No. 5,994,629. Plant cells are further incubated for several days. The expression of the transgene may be tested for at this time. After further incubation for several weeks in selecting medium, calli or plant cells are transferred to regeneration medium and placed in the light. Shoots are transferred to rooting medium and resulting plants are transferred into the glass house.

Alternative methods of plant cell transformation include dipping whole flowers into a suspension of bacteria, growing the plants further into seed formation, harvesting the seeds and germinating them in the presence of a selection agent that allows the growth of the transformed seedlings only. Alternatively, germinated seeds may be treated with a herbicide that only the transformed plants tolerate.

Furthermore, host bacterial species may naturally interact in specific ways with a number of plants. These bacterial-plant interactions are very different from the way Agrobacterium naturally interacts with plants. Thus, the tissues and cells that have are transformable by Agrobacterium may be different in the case of the employment of other bacteria. Some plant cell types that are especially desirable to transform include meristem, pollen and pollen tubes, seed embryos, flowers, ovules, and leaves. Plants that are especially desirable to transform include corn, rice, wheat, soybean, alfalfa and other leguminous plants, potato, tomato, and so on.

### F. Uses of Transformation System

The biological transformation system described here can be used to introduce one or more sequences of interest (transgene) into eukaryotic cells and especially into plant cells.

The following examples are offered by way of illustration, and not by way of limitation.

### EXAMPLES

### Example 1

### Construction of pCAMBIA5105

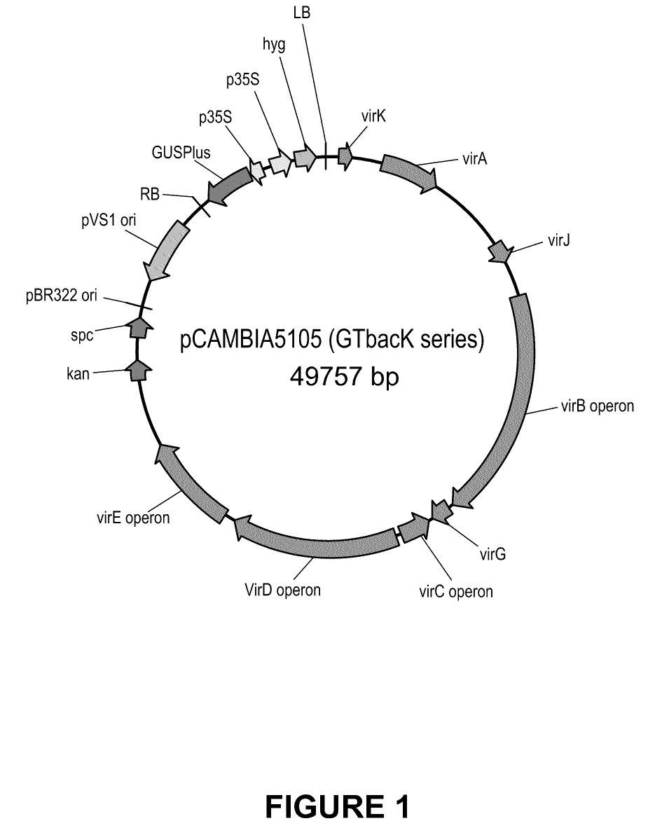

An approximately 40 kb Not I partial fragment was isolated from Ti EHA105 plasmid (a Ti plasmid derived from pTiBO542, but having a precise deletion of T-DNA). The fragment contains the vir regulon from A-E3 as well as some non-essential vir genes. The fragment was modified by adding sequence encoding GUS-Intron within T-borders, the pVS-1 ori and double bacterial selection. A unique restriction site is located outside the left border and unique restriction sites are located within the T-DNA polylinker. A schematic of this vector, called pCAMBIA5105, is shown in FIG. 1. The complete sequence is found in GenBank as Accession No. EF042581 (Version EF042581.1 is incorporated in its entirety).

### Example 2

### Transformation of Rice and Tobacco Using pCAMBIA5105

Transient transformation assays in rice and tobacco using Agrobacterium tumifaciens LBA288 and Sinorhizobium meliloti (Sme) demonstrated that pCAMBIA5105 is capable of excellent quality and quantity of gene transfer. Moreover, stable integration of the DNA in both rice and tobacco is confirmed in both bacterial genera.

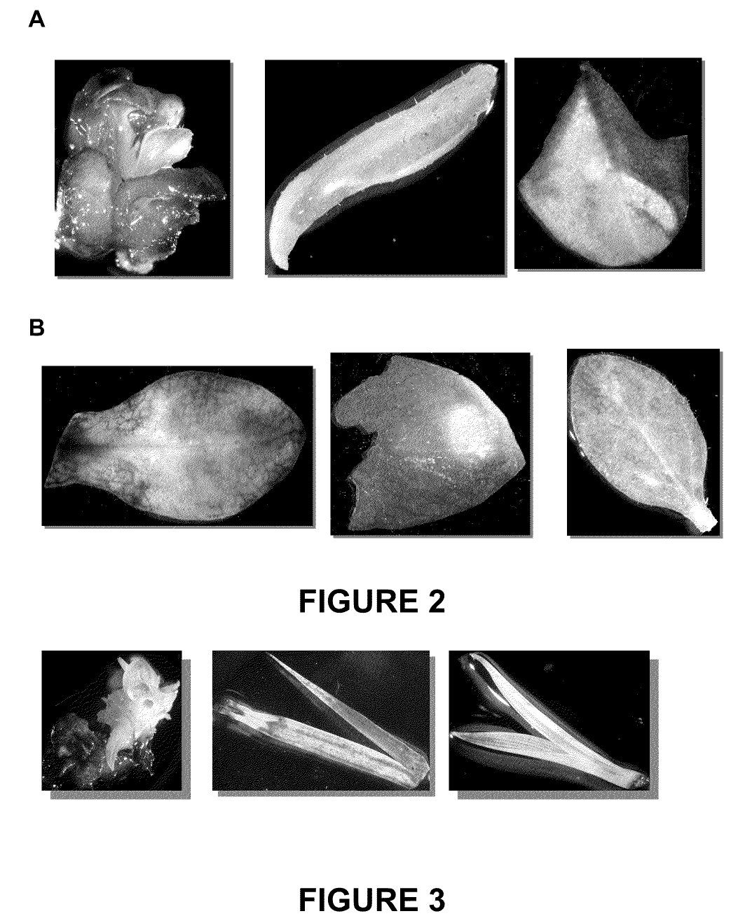

FIG. 2 shows examples of GUS expression patterns in typical transformed tobacco shoots that were transformed with either (A) LBA288 (pCAMBIA5105) or (B) Sme (PCAMBIA 5105). Table 1 presents data of GUS expression in tobacco transformants.

TABLE 1
Stable expression of GUS in tobacco transformants from
LBA288 (pCAMBIA5105) or Sme (pCAMBIA5105).

| | # of shoots positive for | # of positive |
|---|---|---|
| | GUS/total regenerated | plantlets with |
| Bacteria/construct | shoots on Hyg+ plates | roots |
| LBA288 | 86/188 | 13 |
| (pCAMBIA5105) | | |
| Sme | 5/7 | 2 |
| (pCAMBIA5105) | | |

Similar results were obtained from transformation of rice using pCAMBIA5105. FIG. 3 shows examples of GUS expression patterns in typical regenerated rice shoots that were transformed with Sme (pCAMBIA 5105), and transformation data is presented in Table 2.

TABLE 2
Stable expression of GUS in rice transformants
from Sme (pCAMBIA5105).

| | # of shoots positive for | # of positive |
|---|---|---|
| | GUS/total regenerated | plantlets with |
| Bacteria/construct | shoots on Hyg+ plates | roots |
| Sme (pCAMBIA5105) | 7/9 | 7 |

### Example 3

### Transformation Using pCAMBIA5105 in Different Bacterial Species

In these experiments, pCAMBIA5105 was introduced by electroporation into Rhizobium spp ANU240, Rhizobium leguminosarum biovar trifolii strain ANU843, strain ANU845 (derived from ANU843 but psym-), Mesorhizobium loti, Erwinia herbicola, and Pseudomonas fluorescens competent cells, plated on YM or LB media with the appropriate selection, and incubated at 29° C. At various times post incubation, colonies were observed from all transformation experiments except no E. herbicola colonies were observed. Using specifically designed primers, the presence (or absence) of the plasmid and the identity of the bacterial strains were verified by amplification analysis (Table 3).

TABLE 3
Amplification primers

| | | | | |
|---|---|---|---|---|
| | | | SEQ | |
| | | | ID | Product |
| Species/Strain Gene | Primer name | Sequence 5′-3′ | No. | size (bp) |

| | | | | |
|---|---|---|---|---|
| R. leguminosarum | attSpATREV | GGAGAATGGGAGTGACCTGA | 1 | 700 |
| (all strains) | | | | |
| 16SrRNA | R1e16SrRNAFW | CACGTAGGCGGATCGATC | 2 | |
| R. leguminosarum | R1e16SrRNAREV | TTAGCTCACACTCGCGTGCT | 3 | 450 |
| bv. trifolii | | | | |
| NodD | RtrNodDFW | CCTGCAGTTCGCGAAGCTCT | 4 | |
| Rhizobium spp | RtrNodDREV | CGCTTAAAACCGTGCTCCAG | 5 | 350 |
| NGR234 NodD1 | NGR234NodD1FW | GCCAGAAATGTTCATGTCGC | 6 | |
| | | ACA | | |
| M. ciceri/M. loti | NGR234NodD1REV | AATGGGTTGCGGAAGTTCGGT | 7 | 500 |
| 16SrRNA | M1o16SrRNAFW | CCCATCTCTACGGAACAACT | 8 | |
| M. ciceri/M. loti | M1o16SrRNAREV | ACTCACCTCTTCCGGACTCG | 9 | |
| 16 SrRNA | M1opMLRepCREV | CACATGGCAAGCCTCCTCA | 10 | |
| Pseudomonas | Pseud16SFW | GACGGGTGAGTAATGCCTA | 11 | 618 |
| fluorescens WAC1650 | Pseud16SREV | CACTGGTGTTCCTTCCTATA | 12 | |
| 16SrDNA | | | | |
| Erwinia herbicola | ITS-PCRFW | GAAGTCGTAACAAGG | 13 | 500 |
| WAC1664 | ITS-PCRREV | CAAGGCATCCACCGT | 14 | and |
| 16S-23S rRNA | | | | 900 |
| intergenic | | | | |
| transcribed | | | | |
| spacer | | | | |

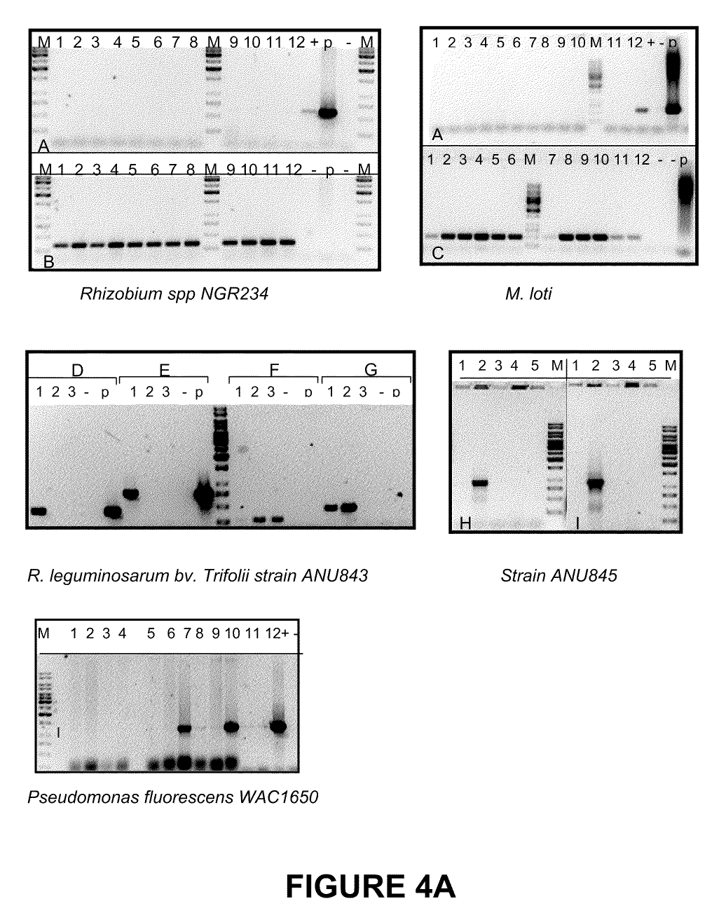

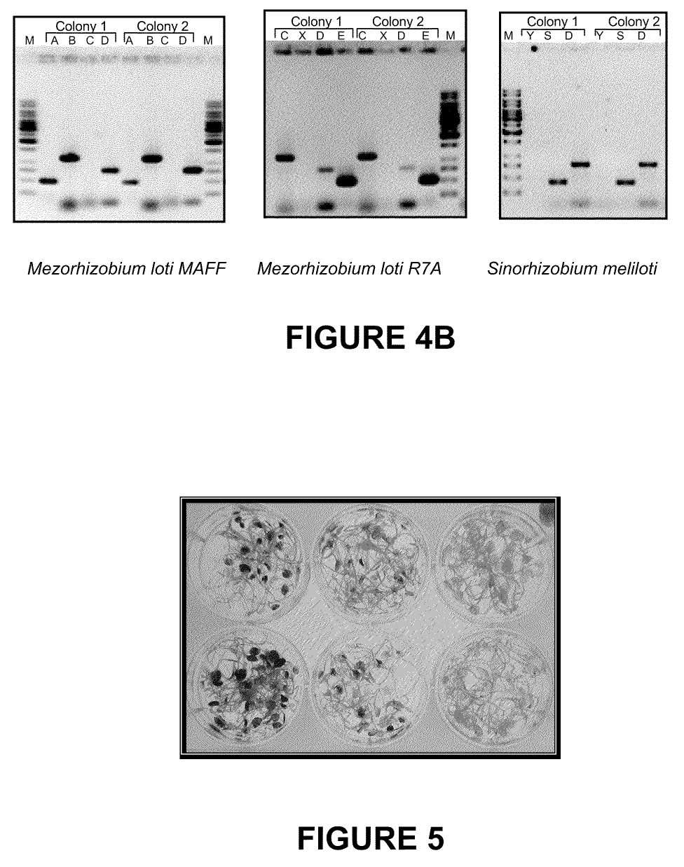

While the predicted genomic fragment indicative of the bacterial strain was found in Rhizobium spp NGR234 and M. loti bacteria colonies, the expected virD4-amplified product, which marks the presence of pCAMBIA5105, was not detected (FIG. 4). These results suggest that the colonies either did not contain pCAMBIA5105 or a very few number of cells hosted the plasmid. Since the colonies were chosen from plates containing the appropriate selections (kanamycin and spectinomycin), the latter possibility seems more likely.

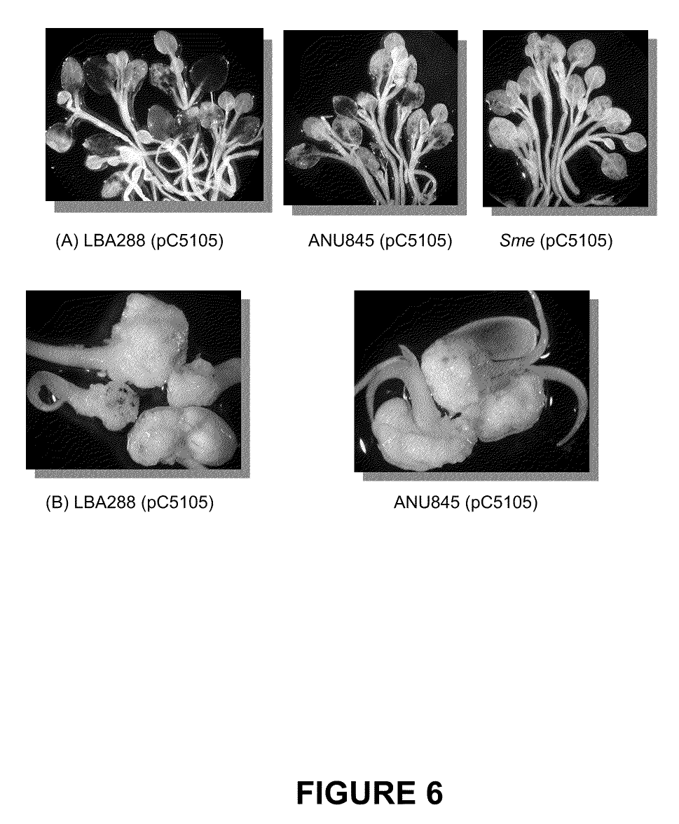

For colonies that were derived from the R. leguminosarum bv. trifolii strains ANU843 and ANU845 plates, the identity of each of the strains as well as the presence of pCAMBIA5105 (FIG. 4A) were confirmed by amplification. Single colonies were selected and tested for their ability to affect DNA transfer to tobacco and rice. Transient assays of transformation of tobacco revealed that both R. leguminosarum strains transfer DNA with comparable efficacies. In addition, the DNA transfer capacity of both ANU843 and ANU845 was comparable to that of LBA288 (FIGS. 5 and 6 and Table 4). Based on the number of blue foci and intensity scores of GUS expression in tobacco seedlings, there was a 7-fold increase in GUS activity in ANU845 (pCAMBIA5105) as compared to Sme (pCAMBIA5105). These results demonstrate the ability of other bacteria species to transfer DNA at similar efficiency level to that of Agrobacterium.

TABLE 4
GUS activity in tobacco seedlings transformed with LBA288
(pCAMBIA5105), ANU845 (pCAMBIA5105), and
Sme (pCAMBIA5105)

| | | # of seedlings | |
|---|---|---|---|
| | | with blue | |
| | Co-cultivation | foci/total # | Intensity |
| Bacteria (constructs) | time | tested (foci #) | score |
| LBA288 (pCAMBIA5105) | 4 days | 35/35 (986) | 6 |
| ANU845 (pCAMBIA5105) | 4 days | 20/20 (944) | 4 |
| Sme (pCAMBIA5105) | 4 days | 23/24 (236) | 2 |
| LBA288 (pCAMBIA5105) | 1 week | 26/26 (894) | 5 |
| ANU845 (pCAMBIA5105) | 1 week | 24/24 (785) | 4 |
| Sme (pCAMBIA5105) | 1 week | 18/25 (101) | 1 |

In addition, tobacco and rice were stably transformed. Assays revealed that R. leguminosarum strains transfer DNA with comparable efficacies. In addition, the DNA transfer capacity of both ANU843 and ANU845 was comparable to that of LBA288 (Table 5). Taken together these results demonstrate the ability of other bacteria species to transfer DNA at similar efficiency level to that of Agrobacterium.

TABLE 5
GUS activity in tobacco and rice stable transformed with LBA288
(pCAMBIA5105), ANU845 (pCAMBIA5105), and
Sme (pCAMBIA5105)

| | | GUS positive lines/#leaf |
|---|---|---|
| | | discs used |
| | | (transformation |
| Transbacter (construct) | Host | efficiency) |
| ANU843 (pCAMBIA5105) | Tobacco W38 | 31/162 (19%) |
| ANU845 (pCAMBIA5105) | | 19/144 (13%) |
| LBA288 (pCAMBIA5105) | | 38/228 (16%) |
| ANU843 (pCAMBIA5105) | Rice Nipponbare | 9/105 (8.6%) |
| ANU845 (pCAMBIA5105) | | 5/120 (4%) |
| LBA288 (pCAMBIA5105) | | 28/235 (16%) |

Extending these results from within the Rhizobiales to the more diverse bacteria such as P. fluorescens (Pfl), pCAMBIA5105 was confirmed present in several Pseudomonas candidate colonies by amplification using specific primers to the kanamycin resistance gene (FIG. 4 P. fluorescens frame). However, in addition to the strongly amplified and expected product, a faint band of identical size is detected in most of the rest of the colonies (FIG. 3 lanes 8, 9, 11, and 12). Such results suggest a differential behavior for the plasmid in the host. Plasmid stability was tested in two colonies, #6 and #25. Cells from each colony were diluted to 10⁻⁷ and 10⁻⁸ and plated on LB Na¹²⁰+MgCl₂¹⁰ (LBNM), LBNM+Km⁵⁰, LBNM+Spec²⁰⁰, or LBNM+Km⁵⁰+Spec²⁰⁰ for two days at 29° C. On day three, the number of colonies on each plate were counted, individual colonies from each of the plates (under the different selections or no selection) were picked, diluted to 10-7 and 10-8 and plated again (second plating) onto the various media as listed above for another two days at 29°. The number of colonies was then counted. Table 6 shows the results from the first and second plating experiments.

TABLE 6
First plating

| | | Pfl | |
|---|---|---|---|
| Selection/construct | Pfl (pC1105.1R) | (pC5105) #6 | Pfl (pC5105) #25 |
| LBNM | 110 | 432 | ~1000 |
| LBNM + Spec²⁰⁰ | 109 | 261 | 522 |
| LBNM + Km⁵⁰ | 0 | 90 | 141 |
| LBNM + Spec²⁰⁰ + | 0 | 72 | ~500 |
| Km⁵⁰ | | | |

Second plating- Pfl (pC1105.1R)

| | LBNM | LBNM + Spec²⁰⁰ |
|---|---|---|
| LBNM | 854 | 667 |
| LBNM + Spec²⁰⁰ | 432 | ~1000 |

| | | | |
|---|---|---|---|
| | | | LBNM + |
| | LBNM + | LBNM + | Spec²⁰⁰ + |
| LBNM | Spec²⁰⁰ | Km⁵⁰ | Km⁵⁰ |

Second plating- Pfl (pCAMBIA5105) #6

| | | | | |
|---|---|---|---|---|
| LBNM | 746 | 35 | 480 | 400 |
| LBNM + Spec²⁰⁰ | 292 | 9 | 321 | 150 |
| LBNM + Km⁵⁰ | 0 | 0 | 6 | 28 |
| LBNM + Spec²⁰⁰ + Km⁵⁰ | 0 | 0 | 7 | 191 |

Second plating- Pfl (pCAMBIA5105) #25

| | | | | |
|---|---|---|---|---|
| LBNM | ~600 | 275 | 700 | 327 |
| LBNM + Spec²⁰⁰ | 85 | 107 | 274 | 207 |
| LBNM + Km⁵⁰ | 0 | 165 | 250 | 123 |
| LBNM + Spec²⁰⁰ + Km⁵⁰ | 0 | 300 | ~500 | 350 |

These results confirm the earlier amplification results and suggest that the two colonies behave differently. In colony 6, the plasmid was lost when plated on either LBNM with or without Spec selection whereas in colony 25, the plasmid was lost when plated on LBNM only. Moreover, in colony 6, plasmid stability was reduced by 100 fold under either Km⁵⁰ or Spec²⁰⁰+Km⁵⁰ whereas in colony 25, it was comparable under the various selections. The effect of vir genes on plasmid stability was studied by including P. fluorescens containing only the pCAMBIA vector, pC1105.1R as a control. Data showed that vir genes have no apparent effect on plasmid stability since pCAMBIA5105 seems to be stable under the Km+Spec selection in both colonies 6 and 25.

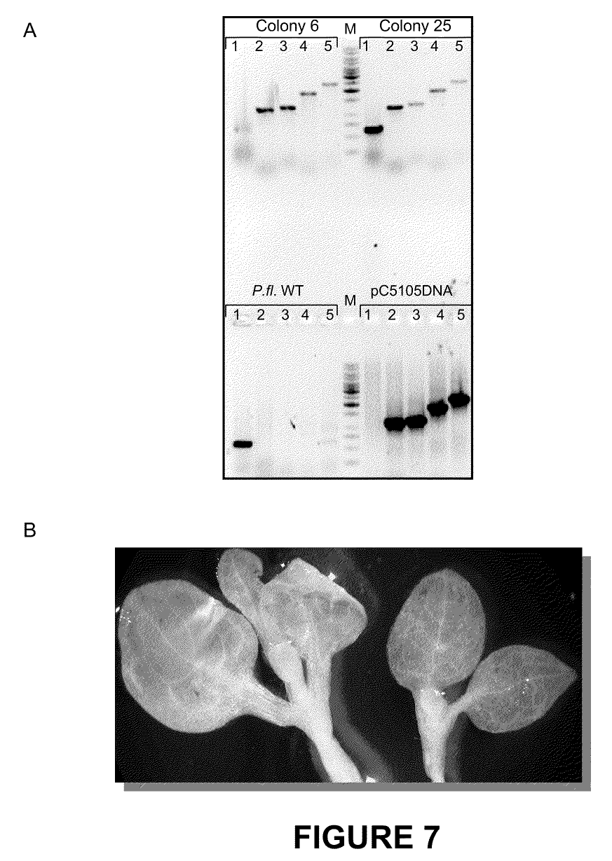

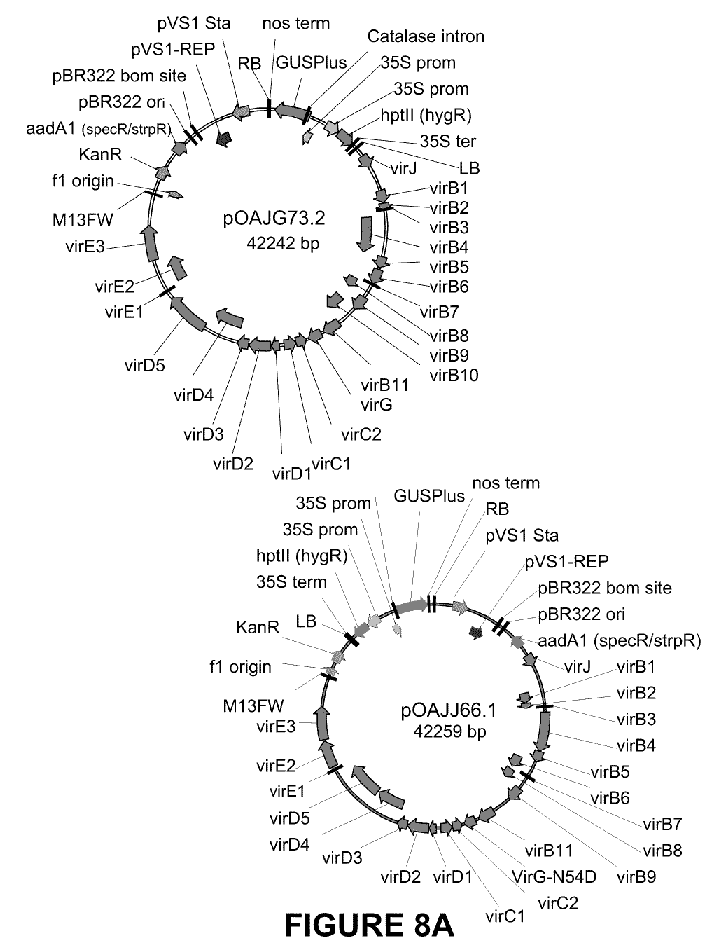

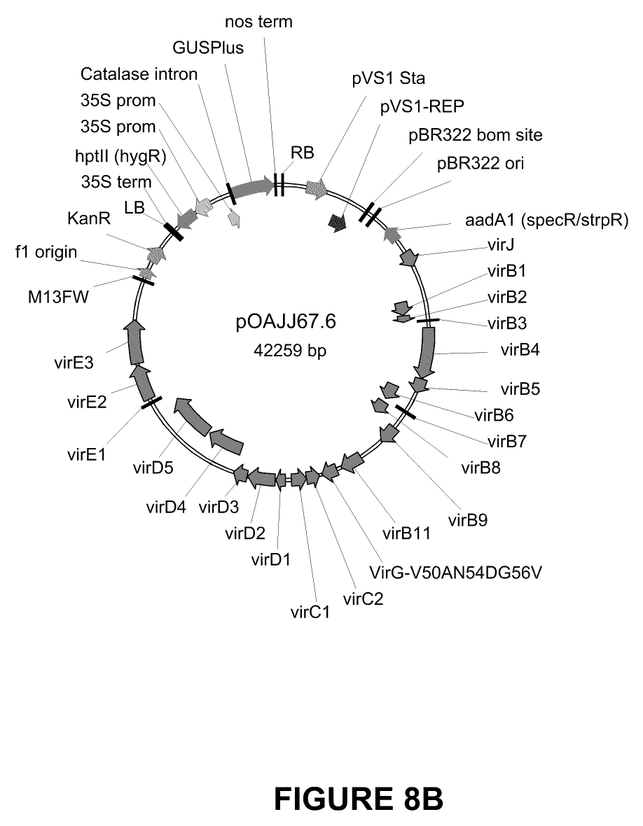

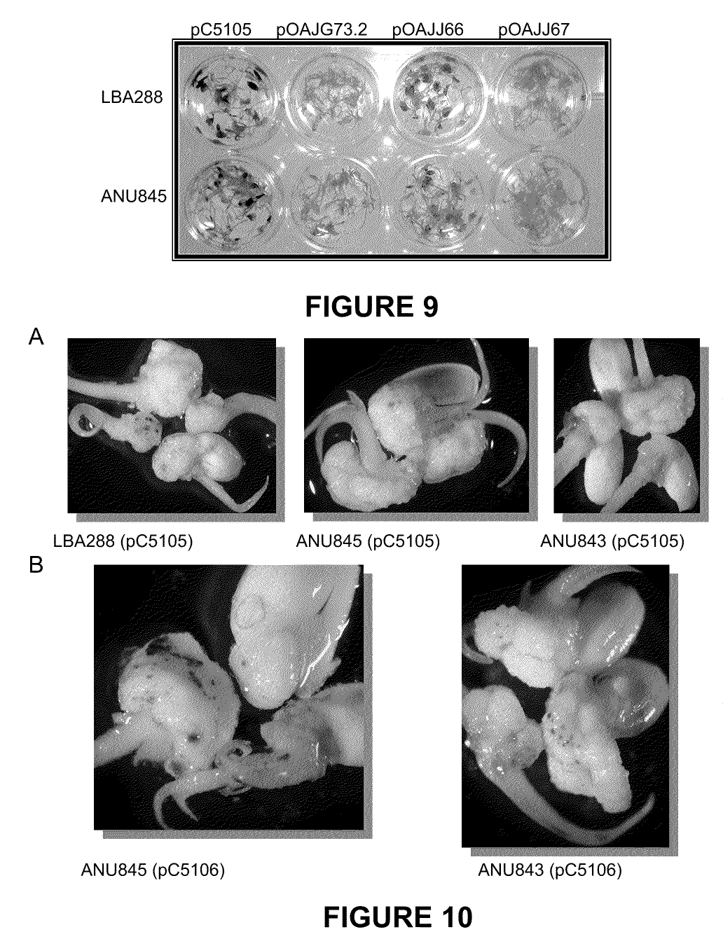

Both colonies were also checked to verify the presence of the plasmid and then tested for their ability to transfer DNA to tobacco cells. FIG. 7A shows the results from amplification analysis and FIG. 7B shows the preliminary results of GUS expression in a few seedlings. The number of blue foci was limited and the bacteria appeared to have affected the health of the seedlings. Tobacco seedlings, which were co-cultivated for 4 or 7 days, had wilted, curled, and the leaves were yellow.

TABLE 7
Summary of GUS activity in tobacco seedlings transformed with
Pseudomonas fluorescens (pC5105) 4 and 7 days post co-cultivation.

| | | # of seedlings | |
|---|---|---|---|
| | Co- | with blue foci/total | |
| | cultivation | # tested | Intensity |
| Bacteria (constructs) | time | (foci #) | score |
| Pseudomonas #6 | 4 days | 0/29 | 0 |
| (pCAMBIA5105) | | | |
| Pseudomonas #25 | 4 days | 1/28 (1) | 0 |
| (pCAMBIA5105) | | | |
| ANU845 (pCAMBIA5105) | 4 days | 27/27 (976) | 5 |
| Pseudomonas #6 | 1 week | 2/46 (13) | 0 |
| (pCAMBIA5105) | | | |
| Pseudomonas #25 | 1 week | 2/35 (4) | 0 |
| (pCAMBIA5105) | | | |
| ANU845 (pCAMBIA5105) | 1 week | 31/31 (1592) | 5 |

### Example 4

### Transformation Using VirG Mutants

In Agrobacterium, the virA and virG genes encode the two key sensory components of the signal transduction and vir gene expression system (Gelvin, 2003). The VirA protein senses an environmental signaling molecule and then modifies the VirG protein bound to it inside the cell. In return, the modified VirG binds to a DNA sequence within the promoters of the vir operons, inducing their expression. The alpha subunit of the E. coli RNA polymerase, however, is unable to interact effectively with the heterologous regulator virG and, therefore, even in the presence of virA and virG, vir gene expression is unsupported in E. coli (Lohrke et al., 1999). A few years ago, Jung et al. (2004) showed that VirG proteins with certain amino acid substitutions are able to effectively interact with the heterologous E. coli RNA polymerase in a virA independent way.

Two specific virG mutants were constructed; virG(N54D) and virG(V50A, N54D, G56A) also known as virG (TM). The virG(N54D) mutant contains an aspartic acid instead of an asparagine at virG amino acid position 54, rendering the expression of VirG protein independent of virA. The mutant protein has ability to stimulate a high level of vir gene expression, especially when expressed from a high-copy-number plasmid. The virG(V50A, N54D, G56A) mutant has a triple amino acid substitution that changes valine-50 to alanine, asparagine-54 to aspartic acid, and glycine-56 to valine. Amino acid position 56 seems to be critical to VirG activity, and glycine at this position abolishes its ability to interact with the alpha-subunit of E. coli.

The wild-type virG was replaced with virG(N54D) or virG(V50A, N54D, G56A) in the virJ-virE3 clone (pOAJE113) that lacks virK and virA genes. These plasmids were then subcloned into pCAMBIA1105.1U, which was used to transform LBA288 and ANU845. For colonies that were derived from the R. leguminosarum bv. trifolii strains ANU843 and ANU845 plates, the identity of each of the strains as well as the presence of pCAMBIA5106 (FIG. 4B) were confirmed by amplification. The bacteria were then tested for their efficacy to transfer DNA to tobacco. Efficacy was assessed initially by transient expression assays.

Tobacco seedlings were infiltrated and co-cultivated with LBA288 or ANU845 containing pCAMBIA5105 (contains virK-E3 region that includes virA), pOAJG73.2 (a virJ-E3 construct that lacks virK and virA but has wild type virG), pOAJJ66 (a virJ-E3 construct that lacks virK and virA and wild type virG was replaced with virG(N54D)), and pOAJJ67 (a virJ-E3 construct that lacks virK and virA and wild type virG was replaced with virG(V50A, N54D, G56A)).

The results in Table 8 demonstrate that virG(N54D) mutant is capable of DNA transfer in a virA independent way in both Agrobacterium and R. leguminosarum bv. trifolii strain ANU845.

TABLE 8
GUS activity in tobacco seedlings transformed with LBA288
(various constructs) 3 and 4 days post co-cultivation or with
ANU845 (various constructs) 7 days post co-cultivation.

| | | # of seedlings | |
|---|---|---|---|
| | Co- | with blue | |
| | cultivation | foci/total # | Intensity |
| Bacteria (constructs) | time | tested (foci #) | score |
| LBA288 | 3 days | 0/18 | — |
| LBA288 (1105.1) | 3 days | 0/16 | — |
| LBA288 (pOAJG73.2) | 3 days | 0/20 | — |
| LBA288 (pOAJJ66.1) | 3 days | 12/14 (151) | 3 |
| LBA288 (pOAJJ66.3) | 3 days | 18/20 (159) | 3 |
| LBA288 (pOAJG73.2 + | 3 days | 20/20 (366) | 5 |
| pRK404tetN54D) | | | |
| N54D mutation was | | | |
| supplied in trans | | | |
| LBA288 (pOAJJ67.6) | 3 days | 0/20 | — |
| LBA288 (pOAJJ67.7) | 3 days | 0/15 | — |
| LBA288 (POAG73.2 + | 3 days | 0/18 | |
| pRK404tetTM) | | | |
| Triple mutation (TM) | | | |
| clone as supplied in trans | | | |
| LBA288 (pC5105) | 3 days | 17/17 (571) | 6 |
| LBA288 | 4 days | 0/20 (0) | — |
| LBA288 (1105.1) | 4 days | 0/20 (0) | — |
| LBA288 (pOAJG73.2) | 4 days | 0/13 (0) | — |
| LBA288 (pOAJJ66.1) | 4 days | 16/16 (294) | 6 |
| LBA288 (pOAJJ66.3) | 4 days | 14/18 (323) | 4 |
| LBA288 (pOAJG73.2 + | 4 days | 20/22 (354) | 6 |
| pRK404tetN54D) | | | |
| LBA288 (pOAJJ67.6) | 4 days | 0/20 (0) | — |
| LBA288 (pOAJJ67.7) | 4 days | 0/20 (0) | — |
| LBA288 (POAG73.2 + | 4 days | 0/18 (0) | |
| pRK404tetTM) | | | |
| LBA288 (pC5105) | 4 days | 13/15 (395) | 6 |
| ANU845 (5105) | 6 days | 31/32 (906) | 5 |
| ANU845 (pOAJG73.2) | 6 days | 0/21 | 0 |
| ANU845 (pOAJJ66.3) | 6 days | 27/27 (552) | 4 |
| ANU845 (pOAJJ67.7) | 6 days | 0 | 0 |

The virG mutants were also tested in rice transformations with ANU845 and ANU843 constructs. Results are shown in Table 9.

TABLE 9
GUS activity in rice seedlings/calli transformed with either ANU845
or ANU843 and various constructs 7 days post co-cultivation.

| | Days | # of seedlings | |
|---|---|---|---|
| Bacterium/ | co- | with blue foci/total | Intensity |
| Construct | cultivation | # tested (foci #) | score |
| ANU843 (pC1105.1U) | 7 days | 0/12 | |
| ANU843 (pC5105) | 7 days | 9/15 (25) | 1 |
| ANU843 (pOAJG73.2) | 7 days | 0/20 | |
| ANU843 (pOAJJ66.3) | 7 days | 11/20 (101) | 2 |
| ANU843 (pOAJJ67.6) | 7 days | 0/15 | |
| ANU845 (pC5105) | 7 days | 7/15 (57) | 2 |
| ANU845 (pOAJG73.2) | 7 days | 0/23 | |
| ANU845 (pOAJJ66.3) | 7 days | 9/15 (168) | 3 |
| ANU845 (pOAJJ67.7) | 7 days | 0/15 | |

These results confirm the ability of a small vir regulon, which lacks virK and virA, to transfer DNA when a single and specific mutation is introduced into virG. These results are in agreement with earlier findings from the literature regarding the increased transformation efficiency using the N54D mutation. In both tobacco and rice, the new unitary vector containing N54D mutation (pCAMBIA5106) has the ability to transfer DNA in both Rhizobium strains ANU843 and ANU845 at efficiency comparable to pC5105 in LBA288 (see Table below and FIG. 10). These results confirm the comparable transformation efficiency between Agrobacterium (pCAMBIA 5106) and ANU 845 (pCAMBIA 5106). Note the improved efficiency of pCAMBIA5106 over that of pCAMBIA 5105.

TABLE 10

| | | GUS positive lines/#leaf |
|---|---|---|
| | | discs used |
| | | (transformation |
| Transbacter (construct) | Host | efficiency) |
| ANU843 (pCAMBIA5106) | Tobacco W38 | 37/148 (25%) |
| ANU845 (pCAMBIA5106) | | 26/108 (24%) |
| LBA288 (pCAMBIA5106) | | 53/313 (17%) |
| ANU843 (pCAMBIA5106) | Rice Nipponbare | 9/130 (7%) |
| ANU845 (pCAMBIA5106) | | 22/147 (15%) |
| LBA288 (pCAMBIA5106) | | 41/230 (18%) |

That capacity however, is abolished or compromised significantly in the virG(V50A, N54D, G56A) mutant or in the wild type construct. Such an outcome would be expected if virG containing the three amino acids substitutions has lost the ability to bind to LBA288 or ANU845 RNA polymerase.

### Example 5

### Replacement of vir Gene Promoters

In this Example, the promoters of the vir genes are replaced with heterologous promoters. Replacing the promoters makes the gene transfer apparatus independent of the VirA-VirG component sensory-signal transduction genetic regulatory system. By this approach, the vir operons can be under control of inducible promoter(s), e.g., “lac” or “tac” promoters. Furthermore, the gene transfer vector is reduced in size.

In one experiment, the native promoters of the virB, virD & virE operons were replaced with the “tac” promoter. Aberrant frequencies and phenotypes of colonies were observed, however. In virtually all the colonies, sequence analysis revealed mutations in the promoter regions of the fusions—generally within the essential −10 and −35 regions of the promoters. This suggests that the tac promoter is too strong when unregulated, and had caused lethality, allowing rare mutants with reduced or negligible expression to emerge in the screen.

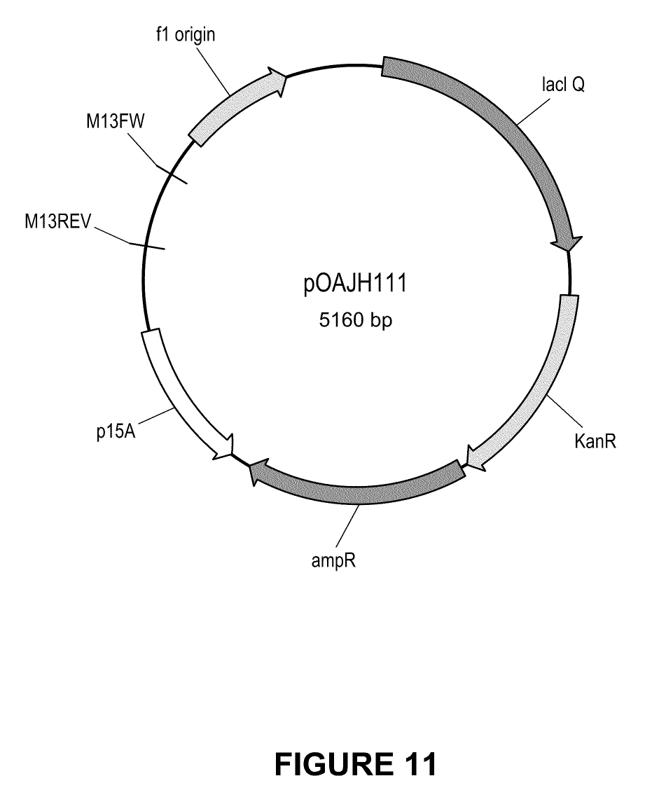

Thus, to avoid leaky expression and potential mutants, a new vector, pOAJH111 (FIG. 11), was constructed which has a low-copy origin of replication (p15A ori, 15-20 copies plasmid/cell) instead of the pUC ori, which is high copy, the lac repressor, lacI^{Q}, and a multi-cloning site (MCS) with rare restriction enzymes to facilitate the cloning of the various vir operons with lacpWT, trp-lac, or any other heterologous promoter.

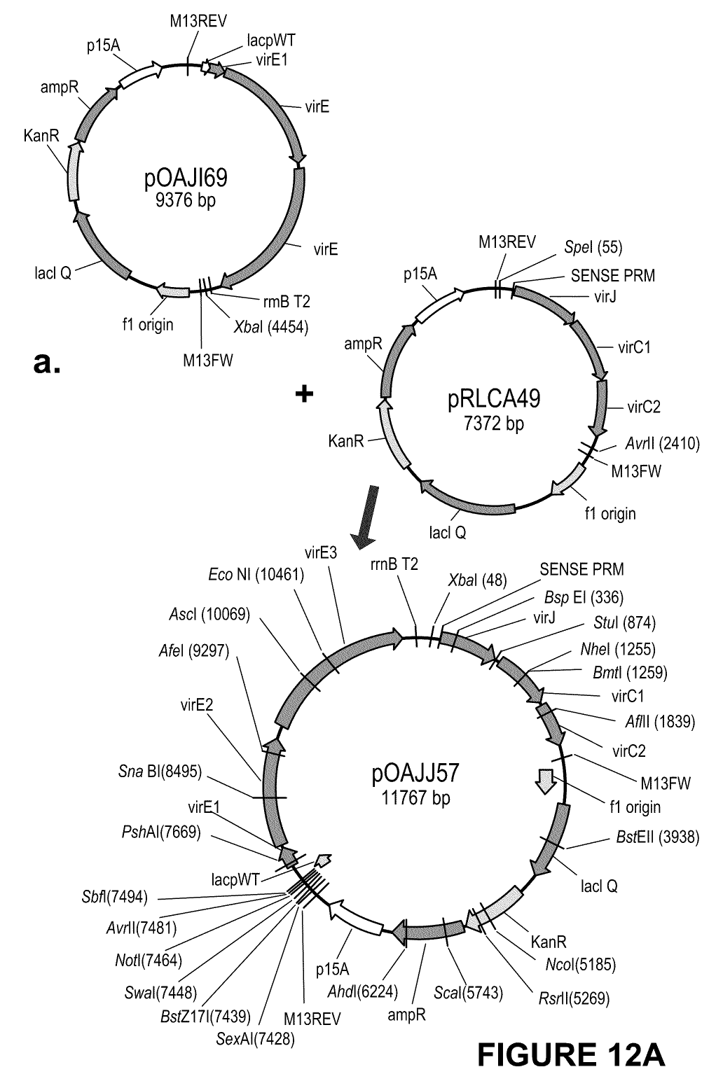

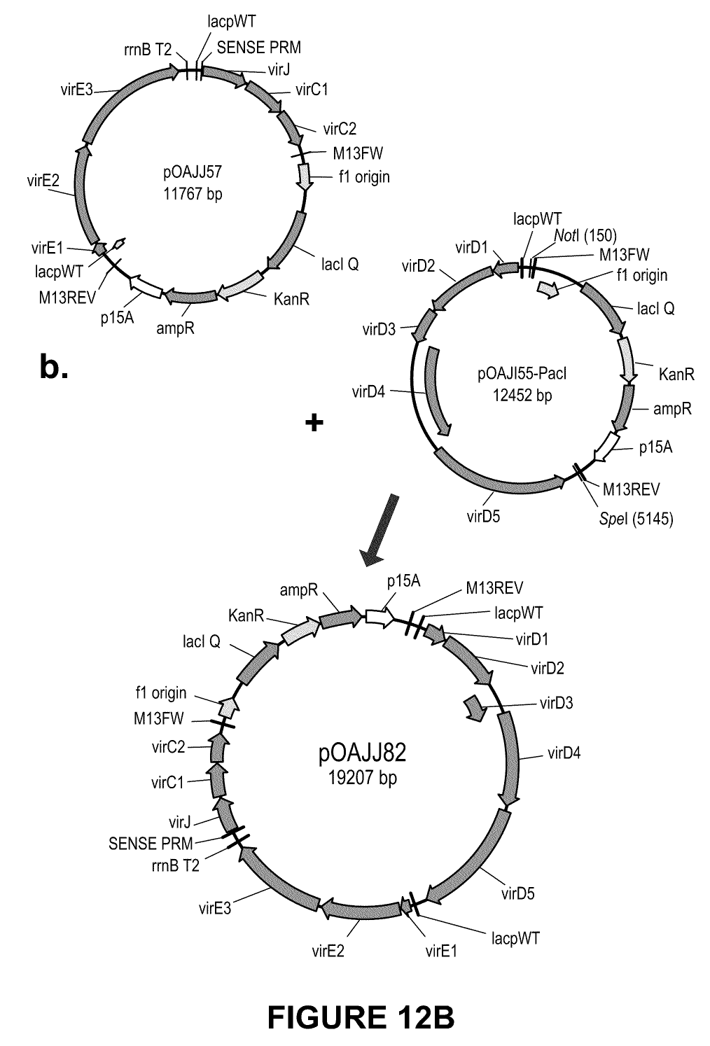

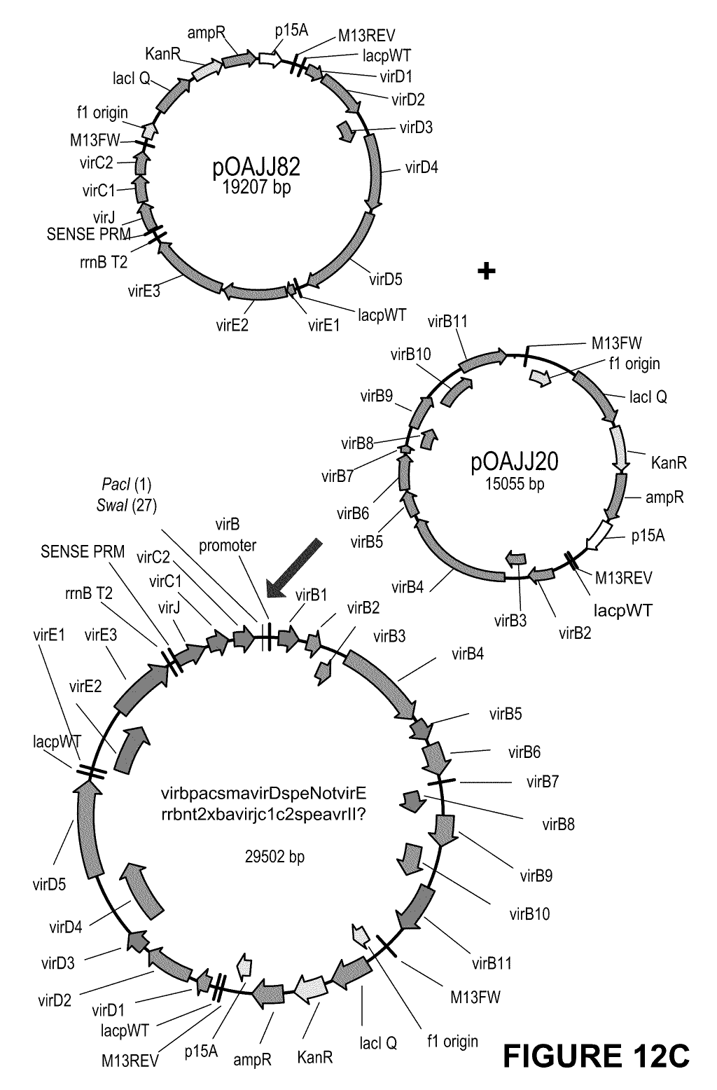

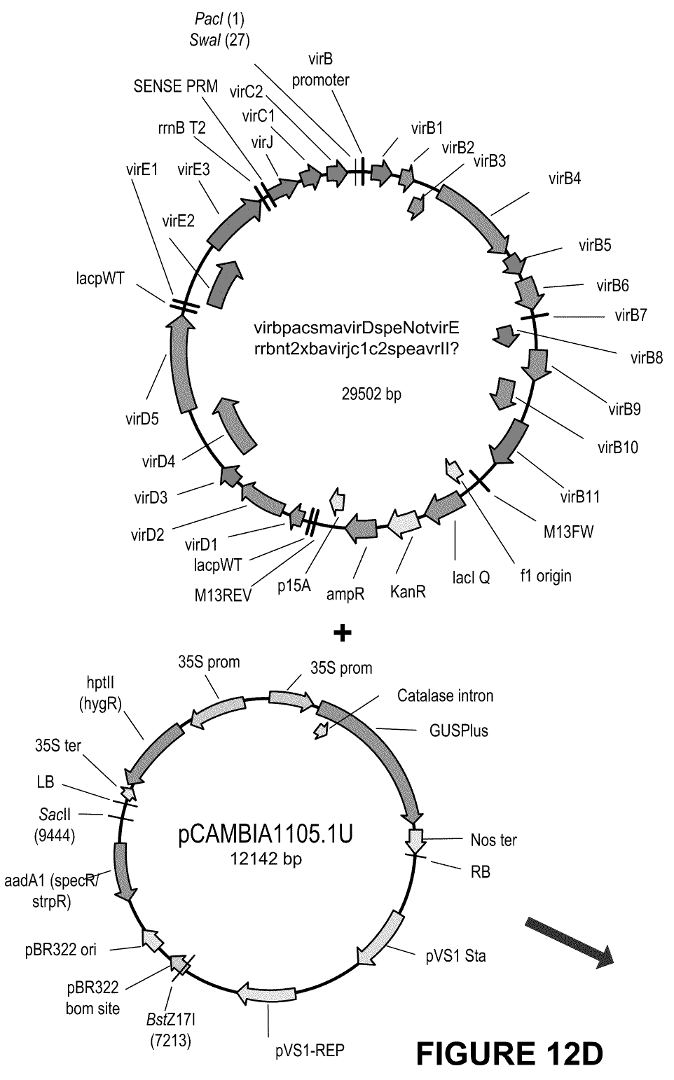

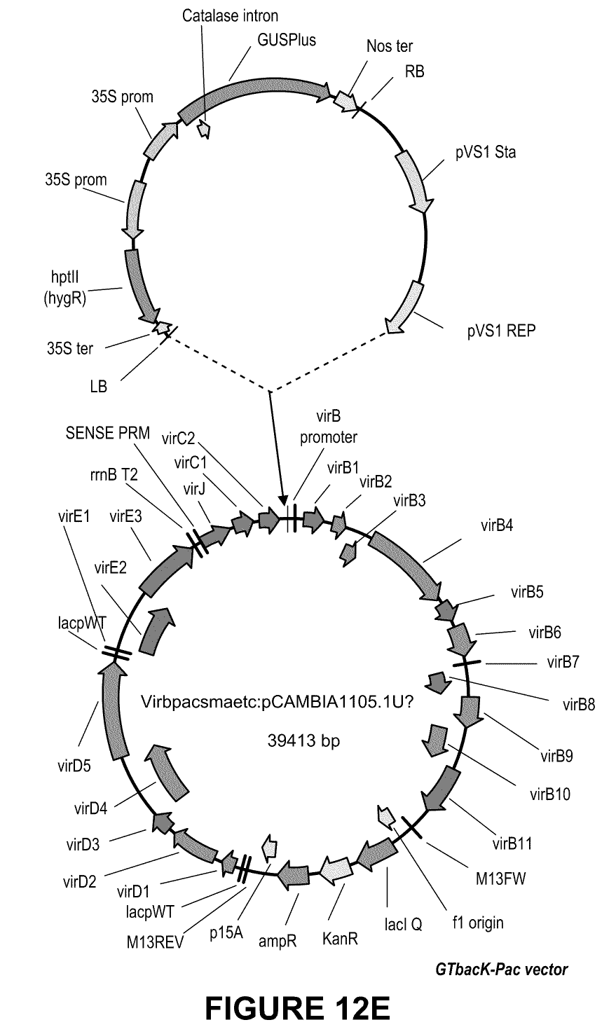

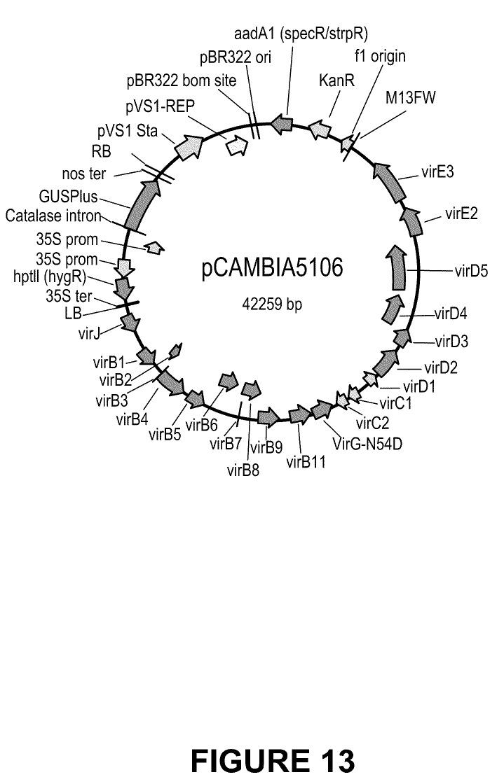

Using pOAJH111, we were able to replace the native vir promoters of the virB, virD & vire operons and the hybrid virJC1C2 operon with either the −64 to +21 “lacpWT” or “tac” promoter that is now under the lacIQ control. Clones obtained were sequenced and except for the tacp-virB operon, all the clones have an intact promoter region and the expected sequences. This regulon will be bounded by a pair of PacI restriction enzyme sites to allow the subcloning of this 25 kb long DNA transfer-enabling cassette into a new vector, pCAMBIA1105.1UP (FIG. 12A).

The four different synthetic operons are characterized by two approaches. The first approach consists of protein profiling after IPTG induction, separating the different proteins within an operon on a gel, blotting them on a membrane, and assaying them by Western blot analysis using specific antibodies raised against selected vir proteins. Realizing that such an approach may be limited by the stoichiometry of the proteins, this approach is being built on by in vivo complementation studies wherein an individual synthetic operon is used in “cis” or “trans” to complement deletion mutants that lack that corresponding operon.

The four vir synthetic operons were expressed by IPTG induction, ran on SDS page gels, blotted and are being assayed with the appropriate controls. As for the complementation studies, the native promoter of the virE operon was replaced with lacpWT virE in the unitary transformation vector pOAJJ66.

Once protein expression is confirmed, the four vir operons are reassembled with the lacI^{Q} gene into a new synthetic “regulon” in pOAJH111, a modified E. coli cloning vector pCR2.1 for ease of cloning, and then that region is moved as a PacI fragment into pCAMBIA1105.1UP, creating pCAMBIA GT-bacKPac (FIG. 12B), as a basis for a new series of unitary plant transformation vectors.

Alternatively wide-host-range promoters, such as those on antibiotic resistance genes from R factors and transposons, e.g. the TetR/TetOA system, will be used, which allows regulated tetracycline resistance in diverse bacteria.

### Example 6

### Transformation with pCAMBIA5016 Deleted for virJ

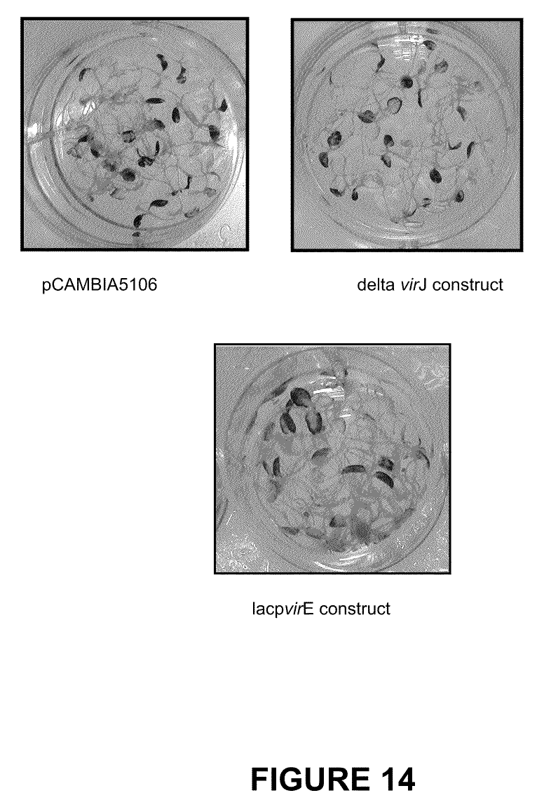

Performance of LBA288 or ANU845 with pC5106, Δ virJ, or lacpvirE constructs was determined in transient assays using tobacco seedlings. FIG. 14 shows results, which confirm the DNA transfer capacity of constructs with a deletion in the virJ or lacp replaced promoter of a vir operon.

### Example 7

### Modified Transformation Protocol

In one modified protocol, a transient gene expression assay uses tobacco seedlings instead of leaf disks. By adding a vacuum infiltration step at the co-cultivation stage, GUS expression was consistently higher than without this step.

In another protocol, a seed-based rice transformation method using Agrobacterium (Toki et al., 2006) is modified to use rice seedling instead of rice calli. Nipponbare seeds, which were germinated at different time intervals on either a) seed plating media (2N6, pH 5.8 with N6 nutrients+glutamine+0.5 g/L proline+1 g/L casamino) or b) Toki's seed plating media (N6D pH 5.8, 2N6 media with 300 mg/L instead of 1 g/L casamino+2.8 g/l instead of 0.5 g/L proline) were used with the following three transformation methods:

Toki's Method (T) that consists of germinating the seeds on N6D for 7 days. On the 7th day, seedlings/calli are mixed with bacterial cells and AAM for 1-2 min then transferred to co-cultivation media with no pre-incubation step.

Standard lab method (Std) that includes seed germination on 2N6 media for 7 days. On the 7th day, the bacterial cells are pre-incubated with AAM media+AS for 1.5 to 2 hours. Afterwards, the seedlings/calli are mixed with bacterial cells and vacuum infiltrated 2-5 min, incubated 10-40 min and transferred to a co-cultivation media.

Modified Toki method (Tm) that consists of seed germination on N6D media for 7 days. On the transformation day, no pre-incubation step is performed, the seedlings/calli are mixed directly with bacterial cells and AAM, vacuum infiltrated, incubated for 10-40 min, and then transferred to co-cultivation media.

In these experiments, 7-day-old seedlings were consistently better material to use than 5 or >10 day-old seedlings. Although the T and Std methods were repeatable with LBA288, these methods could not be applied to Sme. However, when a modified version was developed and tried, repeatable results were obtained with Sme using two co-cultivation media (Table 11).

TABLE 11
Evaluation of the three transient rice transformation methods.

| | oki's co-cultivation media | | | |
|---|---|---|---|---|
| Germination | | | | |
| time/transformation | | | LBA288 (pC5105) | |
| method* | Sme (pC5105) | | N6DAS + | |
| 7-day-old | N6DAS + | N6DAS + | proline + | |
| seedling/calli | proline | p + AAM | p + AAM | N6DAS |
| Toki's (T) | | 0/29^{†} | 5/8 (61) | 17/25 (375) |
| Standard (Std) | 0/5 | 0/21 | 2/6 (3) | 9/43 (74) |
| Toki's modified (Tm) | 7/17 (33) | 4/26 (16) | | |

*germination was at 32° light whereas the co-cultivation was performed at 25° dark. Co-cultivation time was 4 days for LBA288 and 7 days for Sme.
^{†}The ratio represents GUS positive plants over the total number of plants assayed. The number of foci is shown between parentheses.

The modified protocol of the rice seedling transient assay has several advantages over the other two methods as it obviates the need to prepare calli, a process that takes 3-4 weeks. By simply germinating the seeds on calli-inducing media, the seedlings/calli are ready to use between 7-10 days. In addition, using this method, stable transformants from both LBA 288 (pC5105) and Sme (pC5105) were obtained.

TABLE 12
Rice stable transformation experiments using a
modified Toki et al method.

| | # of shoots positive for | # of positive | |
|---|---|---|---|
| | GUS/total | plantlets | Number of |
| | regenerated shoots | with | Nipponbare |
| Bacteria/construct | on Hyg+ plates | roots | seeds used |
| LBA288 (pC5105) | 8/8 | 8 | 50 |
| Sme (pC5105) | 19/48 | 17 | 100 |

From the foregoing it will be appreciated that, although specific embodiments have been described herein for purposes of illustration, various modifications may be made without deviating from the spirit and scope of the invention. Accordingly, the invention is not limited except as by the appended claims.

We claim:

1. A vector for transformation of plant cells, comprising:
(a) virB, virC, virD, and virE operons;
(b) at least one oriT or T-DNA border sequence, operatively linked to
(c) a sequence of interest.

2. The vector according to claim 1, further comprising an origin of replication that is functional in at least one non-Rhizobiales species.

3. The vector according to claim 1, further comprising virA and virG operons.

4. The vector according to claim 1, further comprising virJ operon.

5. The vector according to claim 1, further comprising a mutant virG, wherein the product of the mutant virG functions independent of a product of virA.

6. The vector according to claim 1, wherein the vir operons are controlled by heterologous promoters.

7. The vector according to claim 6, wherein the heterologous promoters are inducible.

8. The vector according to claim 1, wherein the vector is a plasmid.

9. The vector according to claim 1, wherein the vector is pCAMBIA5105.

10. The vector according to claim 1, wherein the vector is pCAMBIA5106.

11. A vector system for transformation of plant cells, comprising:
(a) a first vector comprising at least one oriT or T-DNA border sequence, operatively linked to a sequence of interest; and
(b) a second vector comprising a set of vir operons, wherein the set consists of virB, virC, virD, and virE operons.

12. The vector system according to claim 11, wherein the first vector and the second vector further comprise an origin of replication that is functional in at least one non-Rhizobiales species.

13. The vector system according to claim 11, wherein the second vector further comprises a virA operon and a virG operon.

14. The vector system according to claim 11, wherein the second vector further comprises a virJ operon.

15. The vector according to claim 11, wherein the second vector further comprises a mutant virG, wherein the product of the mutant virG functions independent of a product of virA.

16. The vector according to claim 11, wherein the vir operons are controlled by heterologous promoters.

17. The vector according to claim 16, wherein the heterologous promoters are inducible.

18. The vector according to claim 11, wherein the first and second vectors are plasmids.

19. The vector according to claim 11, wherein the second vector is integrated into a bacterial host chromosome.
